(19) United States
(12) Patent Application Publication
(10) Pub. No.: US 2009/0005396 A1
(43) Pub. Date: Jan. 1, 2009

(54) Use of Pyrazolyl-Pyrimidine Derivatives in the Treatment of Pain

(75) Inventor: Alf Claesson, Sodertalje (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/912,268

(22) PCT Filed: Apr. 25, 2006
(86) PCT No.: PCT/SE06/00476

§ 371 Date: Oct. 22, 2007

(30) Foreign Application Priority Data
Apr. 27, 2005 (SE) 0500959-2

(51) Int. Cl. A61K 31/506; A61P 25/06; A61P 25/00

(52) U.S. Cl. 514/252.19; 514/275

(57) ABSTRACT

This invention relates to the use of a pyrazolyl-pyrimidine of the formula (I). n=0, 1, 2 or 3 (I) in the manufacture of a medicament for use in the treatment or prophylaxis of pain. and to their pharmaceutical formulations and to their methods of use.

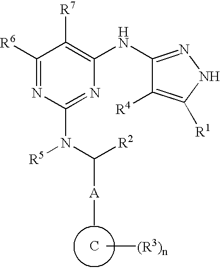

### FIELD OF THE INVENTION

The invention concerns a new use of certain, novel pyrazolyl-pyrimidine derivatives, or pharmaceutically-acceptable salts thereof, which have been found to possess analgesic activity and are accordingly useful in the treatment or prophylaxis of pain conditions in the human or animal body, for example in the manufacture of medicaments for use in the treatment or prevention of pain in a warm-blooded animal such as man.

### BACKGROUND

The current treatment regimes for pain conditions utilise compounds which exploit a very limited range of pharmacological mechanisms. One class of compounds, the opioids, stimulates the endogenous endorphine system; an example from this class is morphine. Compounds of the opioid class have several drawbacks that limit their use, e.g. emetic and constipatory effects and negative influence on respiratory capability. Their use is also restricted because of their addiction liabilities. The second major class of analgesics, the non-steroidal anti-inflammatory analgesics of the COX-1 or COX-2 types, also have liabilities such as insufficient efficacy in severe pain conditions and at long term use the COX-1 inhibitors cause ulcers of the mucosa. Mechanisms of analgesic effects of other currently used medicines are insufficiently characterized and/or have limited therapeutic potential.

Receptor tyrosine kinases (RTKs) are a sub-family of protein kinases that play a critical role in cell signalling and also are involved in a variety of processes related to nerve activity. These include pain transmission in the spinal cord as well as in the peripheral nerve endings where the pain signal starts.

Trk's are the high affinity receptors of the RTK class which are activated by a group of soluble growth factors called neurotrophins (NTFs) among which nerve growth factor (NGF) activates TrkA, brain-derived neurotrophic factor (BDNF) and NT-4/5 activates TrkB and NT3 activates TrkC. Each Trk receptor contains an extra-cellular domain (ligand binding), trans-membrane region and intra-cellular domain (including kinase domain). Upon binding of the ligand, the kinase catalyses the auto-phosphorylation and triggers downstream signal transduction pathways.

Trk's are widely expressed in neuronal tissue during development where they are critical for the maintenance and survival of nerve cells. There are many reports showing that Trk's play important roles in both development and function of the nerve system (e.g. review by Patapoutian, A. et al Current Opinion in Neurobiology, 2001, 11, 272-280).

In the past decade, many scientific reports have been published which link Trk signaling with induction of pain. Levels of NGF are increased after inflammation and NGF contributes to basal and stimulus-induced hyperalgesia (for example, Safieh-Garabedianof et al. British Journal of Pharmacology 1995, 115, 1265). After inflammation BDNF levels are also increased in dorsal root ganglion as indicated by increased mRNA levels (Cho et al. Brain Reseach 1997, 749, 358). Strong support for the involvement of TrkA/TrkB and their ligands NGF/BDNF in pain comes from studies utilizing antibodies towards NGF or fusion proteins of Trk receptors with immunoglobulins which scavenge NGF or BDNF. Several such studies have shown analgesic effects in animals in which inflammation has been induced (for example, Lewin et al. European Journal of Neuroscience 1994, 6, 1903; McMahon et al. Nature Medicine 1995, 1, 774). Although the studies do not deal with the Trk receptor kinases per se they indicate that inhibition of the NGF or BDNF receptor coupled tyrosine kinase may also lead to analgesic effects.

Recent literature also indicates that activation of TrkA with NGF causes downstream upregulation of certain ion channels which are important in increasing the electric signaling from the nerve endings which experience the inflammation, thus inducing pain (for example, VR-1, Winston et al. Pain 2001, 89, 181; sodium channels, Choi et al. Molecular and Cellular Biology 2001, 21, 2695; ASIC, Mamet et al. Journal of Biological Chemistry 2003, 278, 48907).

We have now found surprisingly that certain pyrazolyl-pyrimidine derivatives possess potent analgesic activity by acting as inhibitors of TrkA and TrkB.

There are few reports of selective Trk tyrosine kinase inhibitors that are highly selective for TrkA and TrkB. Cephalon described CEP-751, CEP-701 (George, D. et al Cancer Research, 1999, 59, 2395-2401) and other indolocarbazole analogs (WO0114380) as Trk inhibitors. It was shown that the alkaloid K252a, which is related to CEP-701/751, when injected into rats with pancreatite could suppress mechanical hypersensitivity (Winston et al. Journal of Pain 2003, 4, 329).

It is disclosed in patent application JP 2003-231687 that pyrazole compounds condensed with cycloalkylenes in the 4,5-positions act as neurotrophin receptor inhibitors and can be used as painkillers. GlaxoSmithKIine disclosed certain oxindole compounds as TrkA inhibitors and as useful for the treatment of pain and cancer (WO0220479, WO0220513).

It is disclosed in patent applications WO0250065 and WO0262789 from Vertex Pharmaceuticals that pyrazole compounds are inhibitors of GSK3, Aurora, etc. and are useful for the treatment of cancer. AstraZeneca PLC reported pyrazole compounds as inhibitors of IGF-1 receptor kinase (WO0348133).

### DESCRIPTION OF THE INVENTION

According to one aspect of the invention there is provided the use of a compound of formula (I):

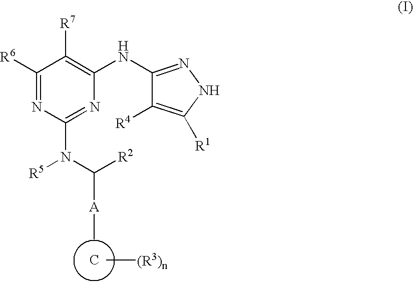

wherein:
A is a direct bond or C₁₋₂alkylene; wherein said C₁₋₂alkylene may be optionally substituted by one or more R²²;
Ring C is carbocyclyl or heterocyclyl;
R¹ and R⁴ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹ and R⁴ independently of each other may be optionally substituted on carbon by one or more R⁸; and wherein if said heterocyclyl contains an —NH— moiety that nitrogen may be optionally substituted by a group selected from R⁹;
R² is selected from hydrogen, cyano, carbamoyl, sulphamoyl, C₁₋₆alkyl, C₂₋₆alkenyl, C₂₋₆alkynyl, C₁₋₆alkoxy, C₁₋₆alkanoyl, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, carbocyclyl or heterocyclyl; wherein R² may be optionally substituted on carbon by one or more R¹⁰; and wherein if said heterocyclyl contains an —NH— moiety that nitrogen may be optionally substituted by a group selected from R¹¹;
R³ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R³ may be optionally substituted on carbon by one or more R¹²; and wherein if said heterocyclyl contains an —NH— moiety that nitrogen may be optionally substituted by a group selected from R¹³;
R⁵ is hydrogen or optionally substituted C₁₋₆alkyl; wherein said optional substituents are selected from one or more R¹⁴;
R⁶ and R⁷ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶;
or R⁶ and R⁷ together with the bond to which they are attached form a 5 or 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸;
n=0, 1, 2 or 3; wherein the values of R³ may be the same or different;
R⁸, R¹⁰, R¹², R¹⁴, R¹⁵, R¹⁷ and R²² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁸, R¹⁰, R¹², R¹⁴, R¹⁵, R¹⁷ and R²² independently of each other may be optionally substituted on carbon by one or more R¹⁹; and wherein if said heterocyclyl contains an —NH— moiety that nitrogen may be optionally substituted by a group selected from R²⁰;
R⁹, R¹¹, R¹³, R¹⁶, R¹⁸ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹¹, R¹³, R¹⁶, R¹⁸ and R²⁰ independently of each other may be optionally substituted on carbon by on or more R²¹;
R¹⁹ and R²¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, NAN-(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹⁹ and R²¹ independently of each other may be optionally substituted on carbon by one or more R²³; and wherein if said heterocyclyl contains an —NH— moiety that nitrogen may be optionally substituted by a group selected from R²⁴;
R¹³ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
R²⁴ is selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
   or a pharmaceutically acceptable salt thereof;
   in the manufacture of a medicament for use in the treatment or prophylaxis of pain.

In a further aspect of the invention there is provided the use of a compound of formula (I) wherein:
A is a direct bond or C₁₋₂alkylene; wherein said C₁₋₂alkylene may be optionally substituted by one or more R²²;
Ring C is carbocyclyl or heterocyclyl;
R¹ and R⁴ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹ and R⁴ independently of each other may be optionally substituted on carbon by one or more R⁸; and wherein if said heterocyclyl contains an —NH— moiety that nitrogen may be optionally substituted by a group selected from R⁹;
R² is selected from hydrogen, cyano, carboxy, carbamoyl, sulphamoyl, C₁₋₆alkyl, C₂₋₆alkenyl, C₂₋₆alkynyl, C₁₋₆alkanoyl, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, carbocyclyl or heterocyclyl; wherein R² may be optionally substituted on carbon by one or more R¹⁰; and wherein if said heterocyclyl contains an —NH— moiety that nitrogen may be optionally substituted by a group selected from R¹¹;
R³ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R³ may be optionally substituted on carbon by one or more R¹²; and wherein if said heterocyclyl contains an —NH— moiety that nitrogen may be optionally substituted by a group selected from R¹³;
R⁵ is hydrogen or optionally substituted C₁₋₆alkyl; wherein said optional substituents are selected from one or more R¹⁴;
R⁶ and R⁷ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶;
or R⁶ and R⁷ together with the bond to which they are attached form a 5 or 6 membered carbocyclic ring or heterocyclic ring wherein said ring is fused to the pyrimidine ring in formula (I); and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸;
n=0, 1, 2 or 3; wherein the values of R³ may be the same or different;
R⁸, R¹⁰, R¹², R¹⁴, R¹⁵, R¹⁷ and R²² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁸, R¹⁰, R¹², R¹⁴, R¹⁵, R¹⁷ and R²² independently of each other may be optionally substituted on carbon by one or more R¹⁹; and wherein if said heterocyclyl contains an —NH— moiety that nitrogen may be optionally substituted by a group selected from R²⁰;
R⁹, R¹¹, R¹³, R¹⁶, R¹⁸ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹¹, R¹³, R¹⁶, R¹⁸ and R²⁰ independently of each other may be optionally substituted on carbon by on or more R²¹;
R¹⁹ and R²¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹⁹ and R²¹ independently of each other may be optionally substituted on carbon by one or more R²³; and wherein if said heterocyclyl contains an —NIH— moiety that nitrogen may be optionally substituted by a group selected from R²⁴;
R²³ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, NiN-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
R²⁴ is selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
   or a pharmaceutically acceptable salt thereof.

Preferred values of the variable groups contained in formula (I) are as follows. Such values may be used, where appropriate, with any of the definitions, claims or embodiments defined hereinbefore or hereinafter.
A is a direct bond.
A is C₁₋₂alkylene.
A is C₁₋₂alkylene optionally substituted by one or more R²².
Ring C is carbocyclyl.
Ring C is heterocyclyl.
Ring C is phenyl or thienyl.
Ring C is phenyl.
Ring C is thienyl.
Ring C is thienyl, pyridyl, thiazolyl.
Ring C is thien-2-yl, pyrid-2-yl, thiazol-2-yl.
Ring C is phenyl or thien-2-yl.
Ring C is phenyl, thienyl, pyridyl, thiazolyl.
Ring C is phenyl, thien-2-yl, pyrid-2-yl, thiazol-2-yl.
Ring C is not pyridyl or isoxazolyl.
Ring C is not pyrid-2-yl, pyrid-3-yl or isoxazol-5-yl.
Ring C and (R³)ₙ together are 4-fluorophenyl.
R¹ is selected from hydrogen, C₁₋₆alkyl, C₁₋₆alkoxy, N,N—(C₁₋₆alkyl)₂-amino, C₁₋₆alkylS(O)ₐ wherein a is 0 or carbocyclyl; wherein R¹ may be optionally substituted on carbon by one or more R⁸; wherein
R⁸ is selected from halo or carbocyclyl.
   R¹ is selected from hydrogen, C₁₋₆alkyl, C₁₋₆alkoxy, N,N—(C₁₋₆alkyl)₂-amino, C₁₋₆alkylS(O)ₐ wherein a is 0 or carbocyclyl.
R¹ is selected from hydrogen, methyl, ethyl, isopropyl, t-butyl, methoxy, ethoxy, propoxy, isopropoxy, sec-butoxy, dimethylamino, methylthio or cyclopropyl; wherein
R⁸ is selected from fluoro, cyclopropyl or phenyl.
R¹ is selected from hydrogen, methyl, ethyl, t-butyl, methoxy, ethoxy, dimethylamino, methylthio or cyclopropyl.
R¹ is selected from hydrogen, methyl, ethyl, isopropyl, t-butyl, trifluoromethyl, cyclopropylmethyl, benzyl, methoxy, ethoxy, propoxy, isopropoxy, sec-butoxy, dimethylamino, methylthio or cyclopropyl.
R¹ is selected from hydrogen, methyl, ethyl, t-butyl, methoxy, dimethylamino, methylthio or cyclopropyl.
R¹ is cyclopropyl.
R⁴ is hydrogen.
R² is C₁₋₆alkyl.
R² is selected from methyl, ethyl or isopropyl.
R² is C₁₋₆alkyl; wherein R² may be optionally substituted on carbon by one or more R¹⁰.
R² is selected from methyl, ethyl or isopropyl; wherein R² may be optionally substituted on carbon by one or more R¹⁰.
R² is C₁₋₆alkyl; wherein R² may be optionally substituted on carbon by one or more R¹⁰;
R¹⁰ is selected from halo, hydroxy, carboxy, amino, C₁₋₆alkoxy, N,N—(C₁₋₆alkyl)₂-amino, C₁₋₆alkanoylamino, N—(C₁₋₆alkyl)carbamoyl, N,N—(C₁₋₆alkyl)₂-carbamoyl or heterocyclyl; wherein R¹⁰ may be optionally substituted on carbon by one or more R¹⁹; and wherein if said heterocyclyl contains an —NH— moiety that nitrogen may be optionally substituted by a group selected from R²⁰;
R¹⁹ is selected from hydroxy or C₁₋₆alkoxy;
R²⁰ is C₁₋₆alkyl.
R² is C₁₋₆alkyl; wherein R² may be optionally substituted on carbon by one or more R¹⁰; wherein
R¹⁰ is selected from hydroxy, carboxy, C₁₋₆alkoxy, N,N—(C₁₋₆alkyl)₂-amino or heterocyclyl; wherein R¹⁰ may be optionally substituted on carbon by one or more R¹⁹; and wherein if said heterocyclyl contains an —NH— moiety that nitrogen may be optionally substituted by a group selected from R²⁰;
   R²⁰ is C₁₋₆alkyl; and
   R¹⁹ is selected from hydroxy or C₁₋₆alkoxy.
R² is selected from methyl, ethyl or isopropyl; wherein R² may be optionally substituted on carbon by one or more R¹⁰;
R¹⁰ is selected from fluoro, hydroxy, carboxy, amino, methoxy, dimethylamino, N-methyl-N-ethylamino, acetylamino, N-methylcarbamoyl, N-ethylcarbamoyl, N,N-dimethylcarbamoyl, pyrrolidin-1-yl, piperazinyl or morpholino; wherein R¹⁰ may be optionally substituted on carbon by one or more R¹⁹; and wherein if said piperazinyl is contains an —NH— moiety that nitrogen may be optionally substituted by a group selected from R²⁰;
   R¹⁹ is selected from hydroxy or methoxy;
   R²⁰ is methyl.
R² is selected from methyl, ethyl or isopropyl; wherein R² may be optionally substituted on carbon by one or more R¹⁰; wherein
R¹⁰ is selected from hydroxy, carboxy, methoxy, N-methyl-N-ethylamino, diethylamino, pyrrolidinyl, piperazinyl or morpholinyl; wherein R¹⁰ may be optionally substituted on carbon by one or more R¹⁹; and wherein said piperazinyl may be optionally substituted on nitrogen by a group selected from R²⁰;
   R²⁰ is methyl; and
   R¹⁹ is selected from hydroxy or methoxy.
R² is selected from methyl, ethyl or isopropyl; wherein R² may be optionally substituted on carbon by one or more R¹⁰; wherein
R¹⁰ is selected from hydroxy, carboxy, methoxy, N-methyl-N-ethylamino, diethylamino, pyrrolidin-1-yl, piperazin-1-yl or morpholino; wherein R¹⁰ may be optionally substituted on carbon by one or more R¹⁹; and wherein said piperazinyl may be optionally substituted on nitrogen by a group selected from R²⁰;
   R²⁰ is methyl; and
   R¹⁹ is selected from hydroxy or methoxy.
   R² is selected from methyl, ethyl, trifluoromethyl, hydroxymethyl, carboxymethyl, aminomethyl, methoxymethyl, morpholinomethyl, 1-hydroxyethyl, 2-hydroxyethyl, 1-carboxyethyl, 2-dimethylaminoethyl, 2-diethylaminoethyl, acetamidomethyl, 2-[N-methyl-N-(2-methoxyethyl)amino]ethyl, 2-[N-methyl-N-(2-hydroxyethyl)amino]ethyl, 2-(N-methylcarbamoyl)ethyl, 2-[N-(2-hydroxyethyl)carbamoyl]ethyl, 2-(N,N-dimethylcarbamoyl)ethyl, 2-morpholinoethyl, 2-pyrrolidin-1-ylethyl or 2-(1-methylpiperazin-4-yl)ethyl, 1-methyl-2-hydroxyethyl.
R² is methyl; wherein R² may be optionally substituted on carbon by one or more R¹⁰; wherein
   R¹⁰ is hydroxy.
R³ is selected from halo, nitro, C₁₋₆alkyl or C₁₋₆alkoxy; wherein R³ may be optionally substituted on carbon by one or more R¹²; wherein
R¹² is halo.
R³ is selected from halo, nitro or C₁₋₆alkoxy.
R³ is selected from fluoro, nitro, methyl or methoxy; wherein R³ may be optionally substituted on carbon by one or more R¹²; wherein
R¹² is fluoro.
R³ is selected from fluoro, nitro, trifluoromethyl or methoxy.
R³ is selected from fluoro, nitro or methoxy.
R³ is fluoro.
R⁵ is hydrogen.
R⁵ is C₁₋₆alkyl.
R⁵ is optionally substituted C₁₋₆alkyl; wherein said optional substituents are selected from one or more R¹⁴.
R⁵ is hydrogen or optionally substituted C₁₋₆alkyl; wherein said optional substituents are selected from one or more R¹⁴; wherein
R¹⁴ is hydroxy.
R⁵ is hydrogen, methyl or optionally substituted ethyl; wherein said optional substituents are selected from one or more R⁴; wherein
R¹⁴ is hydroxy.
R⁵ is hydrogen or optionally substituted ethyl; wherein said optional substituents are selected from one or more R¹⁴; wherein
R¹⁴ is hydroxy.
R⁵ is hydrogen, methyl or 2-hydroxyethyl.
R⁵ is hydrogen or 2-hydroxyethyl.
R⁵ is hydrogen.
R⁶ and R⁷ are independently selected from hydrogen, halo, nitro, cyano, amino, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶.
R⁶ and R⁷ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶.
R⁶ and R⁷ are independently selected from hydrogen, halo, C₁₋₆alkyl, N—(C₁₋₆alkyl)amino, N—(C₁₋₆alkyl)carbamoyl or C₁₋₆alkoxycarbonyl; wherein R⁶ and R⁷ independently of each other may be optionally substituted on carbon by one or more R¹⁵.
R⁶ and R⁷ are independently selected from hydrogen, halo, nitro, cyano, amino, C₁₋₆alkyl, N—(C₁₋₆alkyl)amino, N,N—(C₁₋₆alkyl)₂-amino, N—(C₃₋₆alkyl)carbamoyl, C₁₋₆alkoxycarbonyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶.
R⁶ and R⁷ are independently selected from hydrogen, fluoro, chloro, bromo, methyl, methylamino, ethylamino, propylamino, N-(ethyl)carbamoyl, methoxycarbonyl, ethoxycarbonyl or butoxycarbonyl; wherein R⁶ and R⁷ independently of each other may be optionally substituted on carbon by one or more R¹⁵.
R⁶ and R⁷ are independently selected from hydrogen, fluoro, chloro, bromo, nitro, cyano, amino, methyl, methylamino, ethylamino, propylamino, isopropylamino, dimethylamino, N-methyl-N-propylamino, N-ethylcarbamoyl, methoxycarbonyl, ethoxycarbonyl, butoxycarbonyl, morpholino, pyrrolidinyl or piperazinyl; wherein R⁶ and R⁷ independently of each other may be optionally substituted on carbon by one or more R¹⁵; and wherein said piperazinyl may be optionally substituted on nitrogen by a group selected from R¹⁶.
R⁶ and R⁷ are independently selected from hydrogen, fluoro, chloro, bromo, methyl, ethylamino, propylamino, N-(ethyl)carbamoyl, methoxycarbonyl, ethoxycarbonyl or butoxycarbonyl; wherein R⁶ and R⁷ independently of each other may be optionally substituted on carbon by one or more R¹⁵.
R⁶ and R⁷ together with the bond to which they are attached form a 5 or 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸.
R⁶ and R⁷ together with the bond to which they are attached form a 5 or 6 membered carbocyclic ring or heterocyclic ring wherein said ring is fused to the pyrimidine ring in formula (I); and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸.
R⁶ and R⁷ together with the bond to which they are attached form a 5 or 6 membered carbocyclic ring or heterocyclic ring wherein said ring is fused to the pyrimidine ring in formula (I); and wherein said carbocyclic ring or heterocyclic ring may be optionally substituted on carbon by one or more R¹⁷.
R⁶ and R⁷ together with the pyrimidine to which they are attached form a bicyclic ring selected from quinazolinyl, thieno[3,2-d]pyrimidinyl, thieno[2,3-d]pyrimidinyl, 1H-pyrazolo[3,4-d]pyrimidinyl, thieno[3,4-d]pyrimidinyl, pyrido[2,3-d]pyrimidinyl, 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl; and wherein said bicyclic ring may be optionally substituted on carbon by one or more R¹⁷; and wherein said 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl may be optionally substituted on nitrogen by a group selected from R¹⁸.
R⁶ and R⁷ together with the pyrimidine to which they are attached form quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl; and wherein said quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl may be optionally substituted on carbon by one or more R¹⁷.
R⁶ and R⁷ are independently selected from hydrogen, halo, nitro, cyano, amino, C₁₋₆alkyl, N—(C₁₋₆alkyl)amino, N,N—(C₁₋₆alkyl)₂-amino, N—(C₁₋₆alkyl)carbamoyl, C₁₋₆alkoxycarbonyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶;
or R¹ and R⁷ together with the bond to which they are attached form a 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸.
R⁶ and R⁷ are independently selected from hydrogen, halo, C₁₋₆alkyl, N—(C₁₋₆alkyl)amino, N—(C₁₋₆alkyl)carbamoyl or C₁₋₆alkoxycarbonyl; wherein R⁶ and R⁷ independently of each other may be optionally substituted on carbon by one or more R¹⁵;
or R⁶ and R⁷ together with the bond to which they are attached form a 5 or 6 membered carbocyclic ring or heterocyclic ring wherein said ring is fused to the is pyrimidine ring in formula (I); and wherein said carbocyclic ring or heterocyclic ring may be optionally substituted on carbon by one or more R¹⁷.
R⁶ and R⁷ are independently selected from hydrogen, fluoro, chloro, bromo, nitro, cyano, amino, methyl, methylamino, ethylamino, propylamino, isopropylamino, dimethylamino, N-methyl-N-propylamino, N-ethylcarbamoyl, methoxycarbonyl, ethoxycarbonyl, butoxycarbonyl, morpholino, pyrrolidinyl or piperazinyl; wherein R⁶ and R⁷ independently of each other may be optionally substituted on carbon by one or more R¹⁵; and wherein said piperazinyl may be optionally substituted on nitrogen by a group selected from R¹⁶;
or R⁶ and R⁷ together with the pyrimidine to which they are attached form a bicyclic ring selected from quinazolinyl, thieno[3,2-d]pyrimidinyl, thieno[2,3-d]pyrimidinyl, 1H-pyrazolo[3,4-d]pyrimidinyl, thieno[3,4-d]pyrimidinyl, pyrido[2,3-d]pyrimidinyl, 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl; and wherein said bicyclic ring may be optionally substituted on carbon by one or more R¹⁷; and wherein said 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl may be optionally substituted on nitrogen by a group selected from R¹⁸.
R⁶ and R⁷ are independently selected from hydrogen, fluoro, chloro, bromo, methyl, methylamino, ethylamino, propylamino, N-(ethyl)carbamoyl, methoxycarbonyl, ethoxycarbonyl or butoxycarbonyl; wherein R⁶ and R⁷ independently of each other may be optionally substituted on carbon by one or more R¹⁵;
or R⁶ and R⁷ together with the pyrimidine to which they are attached form quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl; and wherein said quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl may be optionally substituted on carbon by one or more R¹⁷.
R⁶ and R⁷ are independently selected from hydrogen, fluoro, chloro, bromo, methyl, ethylamino, propylamino, N-(ethyl)carbamoyl, methoxycarbonyl, ethoxycarbonyl or butoxycarbonyl; wherein R⁶ and R⁷ independently of each other may be optionally substituted on carbon by one or more R¹⁵;
or R⁶ and R⁷ together with the pyrimidine to which they are attached form quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl; and wherein said quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl may be optionally substituted on carbon by one or more R¹⁷.
R⁶ and R⁷ are independently selected from hydrogen, halo, nitro, cyano, amino, C₁₋₆alkyl, N—(C₁₋₆alkyl)amino, N,N—(C₁₋₆alkyl)₂amino, N—(C₁₋₆alkyl)carbamoyl, C₁₋₆alkoxycarbonyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R₁₆;
or R⁶ and R⁷ together with the bond to which they are attached form a 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸;
R¹⁵ is selected from halo, hydroxy, amino, C₁₋₆alkoxy, N,N—(C₁₋₆alkyl)₂-amino, carbocyclyl or heterocyclyl; wherein R¹⁵ may be optionally substituted on carbon by one or more R¹⁹; and wherein if said heterocyclyl contains an —NH— moiety that nitrogen may be optionally substituted by a group selected from R¹⁸;
R¹⁷ is selected from halo, C₁₋₆alkyl or C₁₋₆alkoxy; wherein R¹⁷ may be optionally substituted on carbon by one or more R¹⁹;
   R¹⁶ is C₁₋₆alkyl;
   R¹⁸ is C₁₋₆alkanoyl;
   R¹⁹ is selected from halo, hydroxy, C₁₋₆alkoxy or heterocyclyl; and wherein if said heterocyclyl contains an —NH— moiety that nitrogen may be optionally substituted by a group selected from R²⁴;
   R²⁰ is C₁₋₆alkyl; and
   R²⁴ is C₁₋₆alkyl.
R⁶ and R⁷ are independently selected from hydrogen, halo, C₁₋₆alkyl, N—(C₁₋₆alkyl)amino, N—(C₁₋₆alkyl)carbamoyl or C₁₋₆alkoxycarbonyl; wherein R⁶ and R⁷ independently of each other may be optionally substituted on carbon by one or more R¹⁵;
or R⁶ and R⁷ together with the bond to which they are attached form a 5 or 6 membered carbocyclic ring or heterocyclic ring wherein said ring is fused to the pyrimidine ring in formula (I); and wherein said carbocyclic ring or heterocyclic ring may be optionally substituted on carbon by one or more R¹⁷; wherein
R¹⁵ is selected from halo, hydroxy, carbocyclyl or heterocyclyl; wherein R¹⁵ may be optionally substituted on carbon by one or more R¹⁹; and wherein if said heterocyclyl contains an —NH— moiety that nitrogen may be optionally substituted by a group selected from R²⁰;
R¹⁷ is selected from halo, C₁₋₆alkyl or C₁₋₆alkoxy; wherein R¹⁷ may be optionally substituted on carbon by one or more R¹⁹;
   R²⁰ is C₁₋₆alkyl;
   R¹⁹ is selected from halo, C₁₋₆alkoxy or heterocyclyl; wherein if said heterocyclyl contains an —NH— moiety that nitrogen may be optionally substituted by a group selected from R²⁴; and
   R²⁴ is C₁₋₆alkyl.
R⁶ and R⁷ are independently selected from hydrogen, fluoro, chloro, bromo, nitro, cyano, amino, methyl, methylamino, ethylamino, propylamino, isopropylamino, dimethylamino, N-methyl-N-propylamino, N-ethylcarbamoyl, methoxycarbonyl, ethoxycarbonyl, butoxycarbonyl, morpholino, pyrrolidinyl or piperazinyl; wherein R⁶ and R⁷ independently of each other may be optionally substituted on carbon by one or more R¹⁵; and wherein said piperazinyl may be optionally substituted on nitrogen by a group selected from R¹⁶;
or R¹⁵ or R⁶ and R⁷ together with the pyrimidine to which they are attached form a bicyclic ring selected from quinazolinyl, thieno[3,2-d]pyrimidinyl, thieno[2,3-d]pyrimidinyl, 1H-pyrazolo[3,4-d]pyrimidinyl, thieno[3,4-d]pyrimidinyl, pyrido[2,3-d]pyrimidinyl, 5,6,7,8-tetrahydro-pyrido[4,3-d]pyrimidinyl, 5,6,7,8-tetrahydro-pyrido[2,3-d]pyrimidinyl or 5,6,7,9-tetrahydro-pyrido[3,4-d]pyrimidinyl; and wherein said bicyclic ring may be optionally substituted on carbon by one or more R¹⁷; and wherein said 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl may be optionally substituted on nitrogen by a group selected from R¹⁵;
R¹⁵ is selected from fluoro, hydroxy, amino, ethoxy, dimethylamino, phenyl, pyrrolidinyl, piperazinyl or morpholino; wherein R¹⁵ may be optionally substituted on carbon by one or more R¹⁹; and wherein said piperazinyl may be optionally substituted on nitrogen by a group selected from R²⁰;
R¹⁷ is selected from fluoro, chloro, methyl, methoxy, ethoxy or propoxy; wherein R¹⁷ may be optionally substituted on carbon by one or more R¹⁹;
   R¹⁶ is methyl;
   R¹⁸ is acetyl;
   R¹⁹ is selected from fluoro, hydroxy, methoxy, piperazinyl, pyrrolidinyl or morpholino; and wherein said piperazinyl may be optionally substituted on nitrogen by a group selected from R²⁴;
   R²⁰ is methyl; and
   R²⁴ is methyl.
R⁶ and R⁷ are independently selected from hydrogen, fluoro, chloro, bromo, methyl, ethylamino, propylamino, N-(ethyl)carbamoyl, methoxycarbonyl, ethoxycarbonyl or butoxycarbonyl; wherein R⁶ and R⁷ independently of each other may be optionally substituted on carbon by one or more R¹⁵;
or R⁶ and R⁷ together with the pyrimidine to which they are attached form quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl; and wherein said quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl may be optionally substituted on carbon by one or more R¹⁷; wherein
R¹⁵ is selected from fluoro, hydroxy, phenyl, piperazinyl, pyrrolidinyl or morpholino; wherein R¹⁵ may be optionally substituted on carbon by one or more R¹⁹; and wherein if said piperazinyl contains an —NH— moiety that nitrogen may be optionally substituted by a group selected from R²⁰;
R¹⁷ is selected from fluoro, chloro, methyl, methoxy or ethoxy; wherein R¹⁷ may be optionally substituted on carbon by one or more R¹⁹;
   R²⁰ is methyl;
   R¹⁹ is selected from fluoro, methoxy, piperazinyl, pyrrolidinyl or morpholino; wherein if said piperazinyl contains an —NH— moiety that nitrogen may be optionally substituted by a group selected from R²⁴; and
   R²⁴ is methyl.
R⁶ and R⁷ are independently selected from hydrogen, chloro, bromo or propylamino; wherein R⁶ and R⁷ independently of each other may be optionally substituted on carbon by one or more R¹⁵; wherein R¹⁵ is hydroxy;
or R⁶ and R⁷ together with the pyrimidine to which they are attached form quinazolinyl.
R¹⁰ is selected from halo, hydroxy, carboxy, amino, C₁₋₆alkoxy, N,N—(C₁₋₆alklyl)₂-amino, C₁₋₆alkanoylamino, N—(C₁₋₆alkyl)carbamoyl, N,N—(C₁₋₆alkyl)₂-carbamoyl or heterocyclyl; wherein R¹⁰ may be optionally substituted on carbon by one or more R¹⁹; and wherein if said heterocyclyl contains an —NH— moiety that nitrogen may be optionally substituted by a group selected from R²⁰.
R¹⁰ is selected from hydroxy, carboxy, C₁₋₆alkoxy, N,N—(C₁₋₆alkyl)₂-amino or heterocyclyl; wherein R¹⁰ may be optionally substituted on carbon by one or more R¹⁹; and wherein if said heterocyclyl contains an —NH— moiety that nitrogen may be optionally substituted by a group selected from R²⁰.
R¹⁰ is selected from fluoro, hydroxy, carboxy, amino, methoxy, dimethylamino, N-methyl-N-ethylamino, acetylamino, N-methylcarbamoyl, N-ethylcarbamoyl, N,N-dimethylcarbamoyl, pyrrolidin-1-yl, piperazinyl or morpholino; wherein R¹⁰ may be optionally substituted on carbon by one or more R¹⁹; and wherein if said piperazinyl contains an —NH— moiety that nitrogen may be optionally substituted by a group selected from R²⁰.
R¹⁰ is selected from hydroxy, carboxy, methoxy, N-methyl-N-ethylamino, diethylamino, pyrrolidinyl, piperazinyl or morpholinyl; wherein R¹⁰ may be optionally substituted on carbon by one or more R¹⁹; and wherein said piperazinyl may be optionally substituted on nitrogen by a group selected from R²⁰.
R¹⁰ is selected from hydroxy, carboxy, methoxy, N-methyl-N-ethylamino, diethylamino, pyrrolidin-1-yl, piperazin-1-yl or morpholino; wherein R¹⁰ may be optionally substituted on carbon by one or more R¹⁹; and wherein said piperazinyl may be optionally substituted on nitrogen by a group selected from R²⁰.
R¹⁴ is hydroxy.
R¹⁵ is selected from halo, hydroxy, carbocyclyl or heterocyclyl; wherein R¹⁵ may be optionally substituted on carbon by one or more R¹⁹; and wherein if said heterocyclyl contains an —NH— moiety that nitrogen may be optionally substituted by a group selected from R²⁰.
R¹⁵ is selected from fluoro, hydroxy, phenyl, piperazinyl, pyrrolidinyl or morpholino; wherein R¹⁵ may be optionally substituted on carbon by one or more R¹⁹; and wherein if said piperazinyl contains an —NH— moiety that nitrogen may be optionally substituted by a group selected from R²⁰.
R¹⁷ is selected from halo, C₁₋₆alkyl or C₁₋₆alkoxy; wherein R¹⁷ may be optionally substituted on carbon by one or more R¹⁹.
R¹⁷ is selected from fluoro, chloro, methyl, methoxy or ethoxy; wherein R¹⁷ may be optionally substituted on carbon by one or more R¹⁹.
   R²⁰ is C₁₋₆alkyl.
   R²⁰ is methyl.
   R¹⁹ is selected from halo, C₁₋₆alkoxy or heterocyclyl; wherein if said heterocyclyl contains an —NH— moiety that nitrogen may be optionally substituted by a group selected from R²⁴.
   R¹⁹ is selected from fluoro, methoxy, piperazinyl, pyrrolidinyl or morpholino; wherein if said piperazinyl contains an —NH— moiety that nitrogen may be optionally substituted by a group selected from R²⁴.
   R¹⁹ is selected from hydroxy or C₁₋₆alkoxy.
   R¹⁹ is selected from hydroxy or methoxy.
   R²⁴ is C₁₋₆alkyl.
   R²⁴ is methyl.
n=0 or 1.
n=0.
n=1.

Therefore in a further aspect of the invention there is provided the use of a compound of formula (I) (as depicted herein above) wherein:
A is a direct bond;
Ring C is carbocyclyl or heterocyclyl;
R¹ is selected from hydrogen, C₁₋₆alkyl, C₁₋₆alkoxy, N,N—(C₁₋₆alkyl)₂-amino, C₁₋₆alkylS(O)ₐ wherein a is 0 or carbocyclyl; wherein R¹ may be optionally substituted on carbon by one or more R⁸;
R² is C₁₋₆alkyl; wherein R² may be optionally substituted on carbon by one or more R¹⁰;
R³ is selected from halo, nitro, C₁₋₆alkyl or C₁₋₆alkoxy; wherein R³ may be optionally substituted on carbon by one or more R¹²;
R⁴ is hydrogen;
R⁵ is hydrogen or optionally substituted C₁₋₆alkyl; wherein said optional substituents are selected from one or more R¹⁴;
R⁶ and R⁷ are independently selected from hydrogen, halo, nitro, cyano, amino, C₁₋₆alkyl, N—(C₁₋₆alkyl)amino, N,N—(C₁₋₆alkyl)₂-amino, N—(C₁₋₆alkyl)carbamoyl, C₁₋₆alkoxycarbonyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶;
or R⁶ and R⁷ together with the bond to which they are attached form a 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸;
R⁸ is selected from halo or carbocyclyl;
R¹⁰ is selected from halo, hydroxy, carboxy, amino, C₁₋₆alkoxy, N,N—(C₁₋₆alkyl)₂-amino, C₁₋₆alkanoylamino, N—(C₁₋₆alkyl)carbamoyl, N,N—(C₁₋₆alkyl)₂-carbamoyl or heterocyclyl; wherein R¹⁰ may be optionally substituted on carbon by one or more R¹⁹; and wherein if said heterocyclyl contains an —NH— moiety that nitrogen may be optionally substituted by a group selected from R²⁰;
R¹² is halo;
R¹⁴ is hydroxy;
R¹⁵ is selected from halo, hydroxy, amino, C₁₋₆alkoxy, N,N—(C₁₋₆alkyl)₂-amino, carbocyclyl or heterocyclyl; wherein R¹⁵ may be optionally substituted on carbon by one or more R¹⁹; and wherein if said heterocyclyl contains an —NH— moiety that nitrogen may be optionally substituted by a group selected from R²⁰;
   R¹⁶ is C₁₋₆alkyl;
   R¹⁷ is selected from halo, C₁₋₆alkyl or C₁₋₆alkoxy; wherein R¹⁷ may be optionally substituted on carbon by one or more R¹⁹;
   R¹⁸ is C₁₋₆alkanoyl;
   R¹⁹ is selected from halo, hydroxy, C₁₋₆alkoxy or heterocyclyl; and wherein if said heterocyclyl contains an —NH— moiety that nitrogen may be optionally substituted by a group selected from 124;
   R²⁰ is C₁₋₆alkyl;
   R²⁴ is C₁₋₆alkyl; and
n=0 or 1.
   or a pharmaceutically acceptable salt thereof.

Therefore in a farther aspect of the invention there is provided the use of a compound of formula (I) (as depicted herein above) wherein:
A is a direct bond;
ing C is carbocyclyl or heterocyclyl;
R¹ is selected from hydrogen, C₁₋₆alkyl, C₁₋₆alkoxy, N,N—(C₁₋₆alkyl)₂-amino, C₁₋₆alkylS(O)ₐ wherein a is 0 or carbocyclyl;
R² is C₁₋₆alkyl; wherein R² may be optionally substituted on carbon by one or more R¹⁰;
R³ is selected from halo, nitro or C₁₋₆alkoxy;
R⁴ is hydrogen;
R⁵ is hydrogen or optionally substituted C₁₋₆alkyl; wherein said optional substituents are selected from one or more R¹⁴;
R⁶ and R⁷ are independently selected from hydrogen, halo, C₁₋₆alkyl, N—(C₁₋₆alkyl)amino, N—(C₁₋₆alkyl)carbamoyl or C₁₋₆alkoxycarbonyl; wherein R⁶ and R⁷ independently of each other may be optionally substituted on carbon by one or more R¹⁵;
or R⁶ and R⁷ together with the bond to which they are attached form a 5 or 6 membered carbocyclic ring or heterocyclic ring wherein said ring is fused to the pyrimidine ring in formula (I); and wherein said carbocyclic ring or heterocyclic ring may be optionally substituted on carbon by one or more R¹⁷;
R¹⁰ is selected from hydroxy, carboxy, C₁₋₆alkoxy, N,N—(C₁₋₆alkyl)₂-amino or heterocyclyl; wherein R¹⁰ may be optionally substituted on carbon by one or more hydroxy or C₁₋₆alkoxy; and wherein if said heterocyclyl contains an —NH— moiety that nitrogen may be optionally substituted by a group selected from R²⁰;
R¹⁴ is hydroxy;
R¹⁵ is selected from halo, hydroxy, carbocyclyl or heterocyclyl; wherein R¹⁵ may be optionally substituted on carbon by one or more R¹⁹; and wherein if said heterocyclyl contains an —NH— moiety that nitrogen may be optionally substituted by a group selected from R²⁰;
R¹⁷ is selected from halo, C₁₋₆alkyl or C₁₋₆alkoxy; wherein R¹⁷ may be optionally substituted on carbon by one or more R¹⁹; wherein R¹⁹ is selected from halo, C₁₋₆alkoxy or heterocyclyl; wherein if said heterocyclyl contains an —NH— moiety that nitrogen may be optionally substituted by a group selected from R²⁴;
   R²⁰ is C₁₋₆alkyl;
   R²⁴ is C₁₋₆alkyl; and
n=0 or 1;
   or a pharmaceutically acceptable salt thereof.

Therefore in a further aspect of the invention there is provided the use of a compound of formula (I) (as depicted herein above) wherein:
A is a direct bond;
Ring C is phenyl, thienyl, pyridyl, thiazolyl;
R¹ is selected from hydrogen, methyl, ethyl, isopropyl, t-butyl, trifluoromethyl, cyclopropylmethyl, benzyl, methoxy, ethoxy, propoxy, isopropoxy, sec-butoxy, dimethylamino, methylthio or cyclopropyl;
R² is selected from methyl, ethyl, trifluoromethyl, hydroxymethyl, carboxymethyl, aminomethyl, methoxymethyl, morpholinomethyl, 1-hydroxyethyl, 2-hydroxyethyl, 1-carboxyethyl, 2-dimethylaminoethyl, 2-diethylaminoethyl, acetamidomethyl, 2-[N-methyl-N-(2-methoxyethyl)amino]ethyl, 2-[N-methyl-N-(2-hydroxyethyl)amino]ethyl, 2-(N-methylcarbamoyl)ethyl, 2-[N-(2-hydroxyethyl)carbamoyl]ethyl, 2-(N,N-dimethylcarbamoyl)ethyl, 2-morpholinoethyl, 2-pyrrolidin-1-ylethyl or 2-(1-methylpiperazin-4-yl)ethyl, 1-methyl-2-hydroxyethyl;
R³ is selected from fluoro, nitro, trifluoromethyl or methoxy;
R⁴ is hydrogen;
R⁵ is hydrogen, methyl or 2-hydroxyethyl;
R⁶ and R⁷ are independently selected from hydrogen, fluoro, chloro, bromo, nitro, cyano, amino, methyl, methylamino, ethylamino, propylamino, isopropylamino, dimethylamino, N-methyl-N-propylamino, N-ethylcarbamoyl, methoxycarbonyl, ethoxycarbonyl, butoxycarbonyl, morpholino, pyrrolidinyl or piperazinyl; wherein R⁶ and R⁷ independently of each other may be optionally substituted on carbon by one or more R¹⁵; and wherein said piperazinyl may be optionally substituted on nitrogen by a group selected from R¹⁶;
or R⁶ and R⁷ together with the pyrimidine to which they are attached form a bicyclic ring selected from quinazolinyl, thieno[3,2-d]pyrimidinyl, thieno[2,3-d]pyrimidinyl, 1H-pyrazolo[3,4-d]pyrimidinyl, thieno[3,4-d]pyrimidinyl, pyrido[2,3-d]pyrimidinyl, 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl; and wherein said bicyclic ring may be optionally substituted on carbon by one or more R¹⁷; and wherein said 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl may be optionally substituted on nitrogen by a group selected from R¹⁸;
R¹⁵ is selected from fluoro, hydroxy, amino, ethoxy, dimethylamino, phenyl, pyrrolidinyl, piperazinyl or morpholino; wherein R¹⁵ may be optionally substituted on carbon by one or more R¹⁹; and wherein said piperazinyl may be optionally substituted on nitrogen by a group selected from R²⁰;
   R¹⁶ is methyl;
R¹⁷ is selected from fluoro, chloro, methyl, methoxy, ethoxy or propoxy; wherein R¹⁷ may be optionally substituted on carbon by one or more R¹⁹;
   R¹⁸ is acetyl;
   R¹⁹ is selected from fluoro, hydroxy, methoxy, piperazinyl, pyrrolidinyl or morpholino; and wherein said piperazinyl may be optionally substituted on nitrogen by a group selected from R²⁴;
   R²⁰ is methyl;
   R²⁴ is methyl;
n=0 or 1;
   or a pharmaceutically acceptable salt thereof.

Therefore in a further aspect of the invention there is provided a the use of a compound of formula (I) (as depicted herein above) wherein:
A is a direct bond;
Ring C is phenyl or thien-2-yl;
R¹ is selected from hydrogen, methyl, ethyl, t-butyl, methoxy, dimethylamino, methylthio or cyclopropyl;
R² is selected from methyl, ethyl or isopropyl; wherein R² may be optionally substituted on carbon by one or more R¹⁰;
R³ is selected from fluoro, nitro or methoxy;
R⁴ is hydrogen;
R⁵ is hydrogen or 2-hydroxyethyl;
R⁶ and R⁷ are independently selected from hydrogen, fluoro, chloro, bromo, methyl, ethylamino, propylamino, N-(ethyl)carbamoyl, methoxycarbonyl, ethoxycarbonyl or butoxycarbonyl; wherein R⁶ and R⁷ independently of each other may be optionally substituted on carbon by one or more R¹⁵;
or R⁶ and R⁷ together with the pyrimidine to which they are attached form quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl; and wherein said quinazolinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl may be optionally substituted on carbon by one or more R¹⁷;
R¹⁰ is selected from hydroxy, carboxy, methoxy, N-methyl-N-ethylamino, diethylamino, pyrrolidin-1-yl, piperazin-1-yl or morpholino; wherein R¹⁰ may be optionally substituted on carbon by one or more hydroxy or methoxy; and wherein said piperazinyl may be optionally substituted on nitrogen by a group selected from R²⁰;
R¹⁵ is selected from fluoro, hydroxy, phenyl, piperazinyl, pyrrolidinyl or morpholino; wherein R¹⁵ may be optionally substituted on carbon by one or more R¹⁹; and wherein if said piperazinyl contains an —NH— moiety that nitrogen may be optionally substituted by a group selected from R²⁰;
R¹⁷ is selected from fluoro, chloro, methyl, methoxy or ethoxy; wherein R¹⁷ may be optionally substituted on carbon by one or more R¹⁹;
   R¹⁹ is selected from fluoro, methoxy, piperazinyl, pyrrolidinyl or morpholino; wherein if said piperazinyl contains an —NH— moiety that nitrogen may be optionally substituted by a group selected from R²⁴;
   R²⁰ is methyl;
   R²⁴ is methyl;
n=0 or 1;
   or a pharmaceutically acceptable salt thereof.

In another aspect of the invention, preferred compounds to be used according to the invention are
5-Chloro-N⁴-(5-cyclopropyl-1H-pyrazol-3-yl)-N²-(1-phenylethyl)pyrimidine-2,4-diamine;
5-Bromo-N⁴-(3-ethyl-1H-pyrazol-5-yl)-N²-(1-phenylethyl)pyrimidine-2,4-diamine;
N⁴-(3-tert-Butyl-1H-pyrazol-5-yl)-5-chloro-N²-(1-phenylethyl)pyrimidine-2,4-diamine;
N⁴-(5-cyclopropyl-1H-pyrazol-3-yl)-N²-(1-phenyl ethyl)-5-(trifluoromethyl)pyrimidine-2,4-diamine;
5-Bromo-N⁴-(5-cyclopropyl-1H-pyrazol-3-yl)-N²-[(1S)-1-(4-fluorophenyl)ethyl]pyrimidine-2,4-diamine;
5-Bromo-N⁴-(5-cyclopropyl-1H-pyrazol-3-yl)-N²-[(1S)-1-phenylpropyl]pyrimidine-2,4-diamine;
5-Bromo-N⁴-(5-cyclopropyl-1H-pyrazol-3-yl)-N²-[(1S)-1-(4-nitrophenyl)ethyl]pyrimidine-2,4-diamine;
(2R)-2-({5-Bromo-4-[(5-cyclopropyl-1H-pyrazol-3-yl)amino]pyrimidin-2-yl}amino)-2-phenylethanol;
5-Bromo-N⁴-(5-cyclopropyl-1H-pyrazol-3-yl)-N²-(1-phenylethyl)pyrimidine-2,4-diamine;
5-Chloro-N⁴-(5-cyclopropyl-1H-pyrazol-3-yl)-N²-(1-phenylpropyl)pyrimidine-2,4-diamine;
5-Chloro-N⁴-(5-cyclopropyl-1H-pyrazol-3-yl)-N²-[(1S)-1-phenylethyl]pyrimidine-2,4-diamine;
5-Chloro-N⁴-(5-cyclopropyl-1H-pyrazol-3-yl)-N²-[(1R)-1-phenylethyl]pyrimidine-2,4-diamine;
5-Bromo-N⁴-(5-cyclopropyl-1H-pyrazol-3-yl)-N²-(1-phenylpropyl)pyrimidine-2,4-diamine;
5-Bromo-N⁴-(5-cyclopropyl-1H-pyrazol-3-yl)-N²-[(1S)-1-phenylethyl]pyrimidine-2,4-diamine;
N⁴-(5-tert-Butyl-1H-pyrazol-3-yl)-5-chloro-N²-[(1S)-1-(4-fluorophenyl)ethyl]pyrimidine-2,4-diamine;
5-Bromo-N⁴-(5-tert-butyl-1H-pyrazol-3-yl)-N²-[(1S)-1-(4-fluorophenyl)ethyl]pyrimidine-2,4-diamine;
5-Bromo-N⁴-(5-cyclopropyl-1H-pyrazol-3-yl)-N²-[(1S)-1-(4-fluorophenyl)ethyl]-6-methylpyrimidine-2,4-diamine;
(2R)-2-({5-Bromo-4-[(5-cyclopropyl-1H-pyrazol-3-yl)amino]-6-methylpyrimidin-2-yl}amino)-2-phenylethanol;
N⁴-(5-cyclopropyl-1H-pyrazol-3-yl)-N²-(1-phenylethyl)pyrimidine-2,4-diamine;
N⁴-(5-cyclopropyl-1H-pyrazol-3-yl)-5-methyl-N²-(1-phenylethyl)pyrimidine-2,4-diamine;
(2S)-2-({5-Bromo-4-[(5-cyclopropyl-1H-pyrazol-3-yl)amino]pyrimidin-2-yl}amino)-2-phenylethanol;
5-Chloro-N⁴-(5-cyclopropyl-1H-pyrazol-3-yl)-N²-[(1S)-1-(4-fluorophenyl)ethyl]pyrimidine-2,4-diamine;
Butyl 6-[(5-cyclopropyl-1H-pyrazol-3-yl)amino]-2-{[1-(4-fluorophenyl)-2-hydroxyethyl]amino}pyrimidine-4-carboxylate;
(2R)-2-({5-Chloro-4-[(5-cyclopropyl-1H-pyrazol-3-yl)amino]pyrimidin-2-yl}amino)-2-(4-fluorophenyl)ethanol;
(2S)-2-({5-Chloro-4-[(5-cyclopropyl-1H-pyrazol-3-yl)amino]pyrimidin-2-yl}amino)-2-(4-fluorophenyl)ethanol;
(2R)-2-({5-Bromo-4-[(5-cyclopropyl-1H-pyrazol-3-yl)amino]pyrimidin-2-yl}amino)-2-(4-fluorophenyl)ethanol;
(2S)-2-({5-Bromo-4-[(5-cyclopropyl-1H-pyrazol-3-yl)amino]pyrimidin-2-yl}amino)-2-(4-fluorophenyl)ethanol;
Methyl 6-[(5-cyclopropyl-1H-pyrazol-3-yl)amino]-2-{[(1R)-1-(4-fluorophenyl)-2-hydroxyethyl]amino}pyrimidine-4-carboxylate;
6-[(5-cyclopropyl-1H-pyrazol-3-yl)amino]-N-[(1R)-1-(4-fluorophenyl)-2-hydroxy ethyl]-2-{[(1R)-1-(4-fluorophenyl)-2-hydroxyethyl]amino}pyrimidine-4-carboxamide;
(2R)-2-({5-Bromo-4-[(5-methyl-1H-pyrazol-3-yl)amino]pyrimidin-2-yl}amino)-2-phenylethanol;
5-Chloro-N²-[(1S)-1-(4-fluorophenyl)ethyl]-N⁴-(5-methyl-1H-pyrazol-3-yl)pyrimidine-2,4-diamine;
5-Bromo-N²-[(1S)-1-(4-fluorophenyl)ethyl]-N⁴-(5-methyl-1H-pyrazol-3-yl)pyrimidine-2,4-diamine;
(2R)-2-({4-[(5-cyclopropyl-1H-pyrazol-3-yl)amino]-5-fluoropyrimidin-2-yl}amino)-2-phenylethanol;
Ethyl 6-[(5-cyclopropyl-1H-pyrazol-3-yl)amino]-2-{[(1S)-1-(4-fluorophenyl)ethyl]amino}pyrimidine-4-carboxylate;
2-({5-Chloro-4-[(5-cyclopropyl-1H-pyrazol-3-yl)amino]pyrimidin-2-yl}amino)-2-(4-fluorophenyl)ethanol;
N⁴-(5-cyclopropyl-1H-pyrazol-3-yl)-N²-[(1S)-1-(4-fluorophenyl)ethyl]-6-methylpyrimidine-2,4-diamine;
2-({5-Bromo-4-[(5-cyclopropyl-1H-pyrazol-3-yl)amino]pyrimidin-2-yl}amino)-2-(4-fluorophenyl)ethanol;
6-Chloro-N⁴-(5-cyclopropyl-1H-pyrazol-3-yl)-N²-[(1S)-1-(4-fluorophenyl)ethyl]pyrimidine-2,4-diamine;
5,6-Dichloro-N⁴-(5-cyclopropyl-1H-pyrazol-3-yl)-N²-[(1S)-1-(4-fluorophenyl)ethyl]pyrimidine-2,4-diamine;
N⁴-(5-cyclopropyl-1H-pyrazol-3-yl)-N²-[(1S)-1-(4-fluorophenyl)ethyl]-5-methylpyrimidine-2,4-diamine;
N⁴-(5-cyclopropyl-1H-pyrazol-3-yl)-5-fluoro-N²-[(1S)-1-(4-fluorophenyl)ethyl]pyrimidine-2,4-diamine;
N⁴-(5-cyclopropyl-1H-pyrazol-3-yl)-N²-[(1S)-1-(4-fluorophenyl)ethyl]pyrimidine-2,4-diamine;
(2S)-2-({5-Chloro-4-[(5-cyclopropyl-1H-pyrazol-3-yl)amino]pyrimidin-2-yl}amino)-2-phenylethanol;
(2R)-2-({5-Chloro-4-[(5-cyclopropyl-1H-pyrazol-3-yl)amino]pyrimidin-2-yl}amino)-2-phenylethanol;
3-({5-Bromo-4-[(5-cyclopropyl-1H-pyrazol-3-yl)amino]pyrimidin-2-yl}amino)-3-(4-fluorophenyl)propanoic acid;
2-[{5-Bromo-4-[(5-cyclopropyl-1H-pyrazol-3-yl)amino]pyrimidin-2-yl}(1-phenylethyl)amino]ethanol;
5-Chloro-N⁴-(5-cyclopropyl-1H-pyrazol-3-yl)-N²-[(1R)-1-(4-fluorophenyl)-2-methoxy ethyl]pyrimidine-2,4-diamine;
5-Chloro-N⁴-(5-cyclopropyl-1H-pyrazol-3-yl)-N²-[1-(4-fluorophenyl)-2-morpholin-4-ylethyl]pyrimidine-2,4-diamine;
2-({5-Chloro-4-[(5-cyclopropyl-1H-pyrazol-3-yl)amino]pyrimidin-2-yl}amino)-2-(2-thienyl)ethanol;
N⁴-(5-cyclopropyl-1H-pyrazol-3-yl)-N²-[(1S)-1-phenylethyl]quinazoline-2,4-diamine;
N⁴-(5-cyclopropyl-1H-pyrazol-3-yl)-N²-[(S)-1-(4-fluorophenyl)ethyl]quinazoline-2,4-diamine;
(2R)-2-({4-[(5-Cyclopropyl-1H-pyrazol-3-yl)amino]quinazolin-2-yl}amino)-2-phenylethanol;
N⁴-(5-tert-Butyl-1H-pyrazol-3-yl)-N²-[(1S)-1-(4-fluorophenyl)ethyl]quinazoline-2,4-diamine;
N²-[(1S)-1-(4-Fluorophenyl)ethyl]-N⁴-(5-methyl-1H-pyrazol-3-yl)quinazoline-2,4-diamine;
(2R)-2-({4-[(5-Methyl-1H-pyrazol-3-yl)amino]quinazolin-2-yl}amino)-2-phenylethanol;
(2R)-2-({4-[(5-Cyclopropyl-1H-pyrazol-3-yl)amino]thieno[3,2-d]pyrimidin-2-yl}amino)-2-phenylethanol;
N⁴-(5-Cyclopropyl-1H-pyrazol-3-yl)-N²-[(1S)-1-(4-fluorophenyl)ethyl]thieno[2,3-d]pyrimidine-2,4-diamine;
N⁴-(5-Cyclopropyl-1H-pyrazol-3-yl)-N²-[(1S)-1-(4-fluorophenyl)ethyl]thieno[3,2-d]pyrimidine-2,4-diamine;
(2R)-2-({4-[(5-Cyclopropyl-1H-pyrazol-3-yl)amino]-1H-pyrazolo[3,4-d]pyrimidin-6-yl}amino)-2-phenylethanol;
(2R)-2-({4-[(5-Cyclopropyl-1H-pyrazol-3-yl)amino]thieno[2,3-d]pyrimidin-2-yl}amino)-2-phenylethanol;
(2R)-2-({4-[(5-Cyclopropyl-1H-pyrazol-3-yl)amino]thieno[3,4-d]pyrimidin-2-yl}amino)-2-phenylethanol;
N⁴-(5-Cyclopropyl-1H-pyrazol-3-yl)-N⁶-[(1S)-1-(4-fluoro phenyl)ethyl]-1H-pyrazolo[3,4-d]pyrimidine-4,6-diamine;
(2R)-2-({4-[(5-Cyclopropyl-1H-pyrazol-3-yl)amino]thieno[3,2-d]pyrimidin-2-yl}amino)-2-(4-fluorophenyl)ethanol;
(3S)-3-({4-[(5-Cyclopropyl-1H-pyrazol-3-yl)amino]thieno[3,2-d]pyrimidin-2-yl}amino)-3-(4-fluorophenyl)propan-1-ol;
N⁴-(5-Cyclopropyl-1H-pyrazol-3-yl)-N²-[(1S)-1-(4-fluorophenyl)ethyl]pyrido[2,3-d]pyrimidine-2,4-diamine;
(2R)-2-({4-[(5-Cyclopropyl-1H-pyrazol-3-yl)amino]pyrido[2,3-d]pyrimidin-2-yl}amino)-2-phenylethanol;
N⁴-(5-Cyclopropyl-1H-pyrazol-3-yl)-N²-[(1S)-1-(4-fluorophenyl)ethyl]-7-methylquinazoline-2,4-diamine;
N⁴-(5-Cyclopropyl-1H-pyrazol-3-yl)-N²-[(1S)-1-(4-fluorophenyl)ethyl]-6-methylquinazoline-2,4-diamine;
N⁴-(5-Cyclopropyl-1H-pyrazol-3-yl)-N²-[(1S)-1-(4-fluorophenyl)ethyl]-6-methoxyquinazoline-2,4-diamine;
7-Chloro-N⁴-(5-cyclopropyl-1H-pyrazol-3-yl)-N²-[(1S)-1-(4-fluorophenyl)ethyl]quinazoline-2,4-diamine;
6-Chloro-N⁴-(5-cyclopropyl-1H-pyrazol-3-yl)-N²-[(1S)-1-(4-fluorophenyl)ethyl]quinazoline-2,4-diamine;
N⁴-(5-Cyclopropyl-1H-pyrazol-3-yl)-N²-[(1S)-1-(4-fluoro phenyl)ethyl]-8-methoxy quinazoline-2,4-diamine;
8-Chloro-N⁴-(5-cyclopropyl-1H-pyrazol-3-yl)-N²-[(1S)-1-(4-fluorophenyl)ethyl]quinazoline-2,4-diamine;
(2R)-2-({4-[(5-Cyclopropyl-1H-pyrazol-3-yl)amino]-1H-pyrazolo[3,4-d]pyrimidin-6-yl}amino)-2-(4-fluorophenyl)ethanol;
(2R)-2-({6-Chloro-4-[(5-cyclopropyl-1H-pyrazol-3-yl)amino]quinazolin-2-yl}amino)-2-phenylethanol;
(2R)-2-({7-Chloro-4-[(5-cyclopropyl-1H-pyrazol-3-yl)amino]quinazolin-2-yl}amino)-2-phenylethanol;
N⁴-(5-Cyclopropyl-1H-pyrazol-3-yl)-7-fluoro-N²-[(1S)-1-(4-fluorophenyl)ethyl]quinazoline-2,4-diamine;
(2R)-2-({4-[(5-Cyclopropyl-1H-pyrazol-3-yl)amino]-7-fluoroquinazolin-2-yl}amino)-2-(4-fluorophenyl)ethanol;
(2R)-2-({4-[(5-Cyclopropyl-1H-pyrazol-3-yl)amino]-7-methylquinazolin-2-yl}amino)-2-(4-fluorophenyl)ethanol;
(2R)-2-({4-[(5-Cyclopropyl-1H-pyrazol-3-yl)amino]-6-methoxyquinazolin-2-yl}amino)-2-(4-fluorophenyl)ethanol;
N⁴-(5-Cyclopropyl-1H-pyrazol-3-yl)-6-fluoro-N²-[(1S)-1-(4-fluorophenyl)ethyl]quinazoline-2,4-diamine;
(2R)-2-({5-Bromo-4-[(3-methoxy-1H-pyrazol-5-yl)amino]pyrimidin-2-yl}amino)-2-(4-fluorophenyl)ethanol;
(2R)-2-[(5-Chloro-4-{[5-(methylthio)-1H-pyrazol-3-yl]amino}pyrimidin-2-yl)amino]-2-(4-fluorophenyl)ethanol;
(2R)-2-({4,5-Dichloro-6-[(5-cyclopropyl-1H-pyrazol-3-yl)amino]pyrimidin-2-yl}amino)-2-(4-fluorophenyl)ethanol;
(2R)-2-{[5-Chloro-4-(1H-pyrazol-5-ylamino)pyrimidin-2-yl]amino}-2-(4-fluorophenyl)ethanol;
(2R)-2-[(5-Chloro-4-{[3-(dimethylamino)-1H-pyrazol-5-yl]amino}pyrimidin-2-yl)amino]-2-(4-fluorophenyl)ethanol;
3-({5-Bromo-4-[(5-cyclopropyl-1H-pyrazol-3-yl)amino]pyrimidin-2-yl}amino)-2-methyl-3-phenylpropanoic acid;
3-({5-Bromo-4-[(5-cyclopropyl-1H-pyrazol-3-yl)amino]pyrimidin-2-yl}amino)-2-methyl-3-phenylpropan-1-ol;
5-Chloro-N⁴-(5-cyclopropyl-1H-pyrazol-3-yl)-N²-[(1S)-1-(4-fluorophenyl)-3-morpholin-4-ylpropyl]pyrimidine-2,4-diamine;
5-Chloro-N⁴-(5-cyclopropyl-1H-pyrazol-3-yl)-N²-[(1S)-1-(4-fluorophenyl)-3-pyrrolidin-1-ylpropyl]pyrimidine-2,4-diamine;
5-Chloro-N⁴-(5-cyclopropyl-1H-pyrazol-3-yl)-N²-[(1S)-3-(diethylamino)-1-(4-fluoro phenyl)propyl]pyrimidine-2,4-diamine;
5-Chloro-N⁴-(5-cyclopropyl-1H-pyrazol-3-yl)-N²-[(1S)-1-(4-fluorophenyl)-3-(4-methyl piperazin-1-yl)propyl]pyrimidine-2,4-diamine;
5-Chloro-N⁴-(5-cyclopropyl-1H-pyrazol-3-yl)-N²-{(1S)-1-(4-fluorophenyl)-3-[(2-methoxyethyl)(methyl)amino]propyl}pyrimidine-2,4-diamine;
2-[[(3S)-3-({5-Chloro-4-[(5-cyclopropyl-1H-pyrazol-3-yl)amino]pyrimidin-2-yl}amino)-3-(4-fluorophenyl)propyl](methyl)amino]ethanol;
(3S)-3-({5-Chloro-4-[(5-cyclopropyl-1H-pyrazol-3-yl)amino]pyrimidin-2-yl}amino)-3-(4-fluorophenyl)propan-1-ol;
(2R)-2-({4-[(5-Cyclopropyl-1H-pyrazol-3-yl)amino]-5-methylpyrimidin-2-yl}amino)-2-(4-fluorophenyl)ethanol;
(2R)-2-({4-[(5-Cyclopropyl-1H-pyrazol-3-yl)amino]-5-fluoropyrimidin-2-yl}amino)-2-(4-fluorophenyl)ethanol;
(R)-2-[4-(5-Cyclopropyl-1H-pyrazol-3-ylamino)-pyrido[2,3d]pyrimidin-2-ylamino]-2-(4-fluoro-phenyl)-ethanol;
5-Chloro-N⁴-(5-cyclopropyl-1H-pyrazol-3-yl)-N²-[(S)-1-(2-methoxy-phenyl)-ethyl]-pyrimidine-2,4-diamine;
(2R)-2-({4-[(5-Cyclopropyl-1H-pyrazol-3-yl)amino]-5-nitropyrinlidin-2-yl}amino)-2-(4-fluorophenyl)ethanol;
N⁴-(5-Cyclopropyl-1H-pyrazol-3-yl)-N²-[(1S)-1-(4-fluorophenyl)ethyl]-5-nitropyrimidine-2,4-diamine;
5-Chloro-N⁴-(5-cyclopropyl-1H-pyrazol-3-yl)-N²-methyl-N²-(1-pyridin-2-ylethyl)pyrimidine-2,4-diamine;
1-({5-Chloro-4-[(5-cyclopropyl-1H-pyrazol-3-yl)amino]pyrimidin-2-yl}amino)-1-phenylpropan-2-ol;
5-Chloro-N²-[(1S)-1-(4-fluorophenyl)-ethyl]-N⁴-(5-trifluoromethyl-1H-pyrazol-3-yl)-pyrimidine-2,4-diamine;
5-Bromo-N⁴-(5-cyclopropyl-1H-pyrazol-3-yl)-N²-(1-pyridin-2-ylethyl)-pyrimidine-2,4-diamine;
N⁴-(5-Benzyl-2H-pyrazol-3-yl)-5-chloro-N²-[(1S)-1-(4-fluorophenyl)ethyl]-pyrimidine-2,4-diamine;
5-Chloro-N²-[(1S)-1-(4-fluorophenyl)-ethyl]-N⁴-(5-isopropyl-2H-pyrazol-3-yl)-pyrimidine-2,4-diamine;
5-Chloro-N⁴-(5-cyclopropylmethyl-1H-pyrazol-3-yl)-N²-[(1S)-1-(4-fluorophenyl)ethyl]-pyrimidine-2,4-diamine;
5-Chloro-N⁴-[5-(cyclopropylmethoxy)-1H-pyrazol-3-yl]-N²-[(1S)-1-(4-fluorophenyl)ethyl]pyrimidine-2,4-diamine;
5-Bromo-N⁴-(5-cyclopropyl-1H-pyrazol-3-yl)-N²-[(1S)-1-(4-trifluoromethyl-thiazol-2-yl)-ethyl]-pyrimidine-2,4-diamine;
N⁴-(5-Cyclopropyl-1H-pyrazol-3-yl)-N²-(1-thiazol-2-yl-ethyl)-pyrimidine-2,4-diamine;
(2R)-2-({4-[(3-sec-Butoxy-1H-pyrazol-5-yl)amino]-5-chloropyrimidin-2-yl}amino)-2-(4-fluorophenyl)ethanol;
(2R)-2-({5-Chloro-4-[(3-propoxy-1H-pyrazol-5-yl)amino]pyrimidin-2-yl}amino)-2-(4-fluorophenyl)ethanol;
(2R)-2-({5-Chloro-4-[(3-isopropoxy-1H-pyrazol-5-yl)amino]pyrimidin-2-yl}amino)-2-(4-fluorophenyl)ethanol;
(2R)-2-({5-Chloro-4-[(3-ethoxy-1H-pyrazol-5-yl)amino]pyrimidin-2-yl}amino)-2-(4-fluorophenyl)ethanol;
5-Chloro-N⁴-(5-cyclopropyl-1H-pyrazol-3-yl)-N²-[(1S)-3-(dimethylamino)-1-(4-fluoro phenyl)propyl]pyrimidine-2,4-diamine;
(3S)-3-({5-Chloro-4-[(5-cyclopropyl-1H-pyrazol-3-yl)amino]pyrimidin-2-yl}amino)-3-(4-fluorophenyl)-N,N-dimethylpropanamide;
(3S)-3-({5-Chloro-4-[(5-cyclopropyl-1H-pyrazol-3-yl)amino]pyrimidin-2-yl}amino)-3-(4-fluorophenyl)-N-methylpropanamide;
3-({5-Chloro-4-[(5-cyclopropyl-1H-pyrazol-3-yl)amino]pyrimidin-2-yl}amino)-3-(2-fluorophenyl)propan-1-ol;
(3S)-3-({5-Chloro-4-[(5-cyclopropyl-1H-pyrazol-3-yl)amino]pyrimidin-2-yl}amino)-3-(4-fluorophenyl)-N-(2-hydroxyethyl)propanamide;
3-({5-Chloro-4-[(5-cyclopropyl-1H-pyrazol-3-yl)amino]pyrimidin-2-yl}amino)-3-(2-methoxyphenyl)propan-1-ol;
3-({5-Chloro-4-[(5-cyclopropyl-1H-pyrazol-3-yl)amino]pyrimidin-2-yl}amino)-3-(2-thienyl)propan-1-ol;
5-Chloro-N⁴-(5-cyclopropyl-1H-pyrazol-3-yl)-N²-[(1R)-1-(4-fluorophenyl)-2-morpholin-4-ylethyl]pyrimidine-2,4-diamine;
(2R)-2-({5-Fluoro-4-[(5-isopropoxy-1H-pyrazol-3-yl)amino]pyrimidin-2-yl}amino)-2-(4-fluorophenyl)ethanol;
N-[(2R)-2-({4-[(5-Cyclopropyl-1H-pyrazol-3-yl)amino]-5-chloropyrimidin-2-yl}amino)-2-(4-fluorophenyl)ethyl]acetamide;
(2R)-2-({4-[(5-Ethoxy-1H-pyrazol-3-yl)amino]-5-fluoropyrimidin-2-yl}amino)-2-(4-fluorophenyl)ethanol;
(3S)-3-({4-[(5-Cyclopropyl-1H-pyrazol-3-yl)amino]-5-fluoropyrimidin-2-yl}amino)-3-(4-fluorophenyl)propan-1-ol;
N²-[(1R)-2-Amino-1-(4-fluorophenyl)ethyl]-5-chloro-N⁴-(5-cyclopropyl-1H-pyrazol-3-yl)pyrimidine-2,4-diamine;
N⁴-(5-Cyclopropyl-1H-pyrazol-3-yl)-N²-[(1S)-1-(4-fluorophenyl)ethyl]pyrimidine-2,4,5-triamine;
(2R)-2-({5-Amino-4-[(5-cyclopropyl-1H-pyrazol-3-yl)amino]pyrimidin-2-yl}amino)-2-(4-fluorophenyl)ethanol;
4-[(5-Cyclopropyl-1H-pyrazol-3-yl)amino]-2-{[(1S)-1-(4-fluorophenyl)ethyl]amino}pyrimidine-5-carbonitrile;
5-Chloro-N⁴-(5-cyclopropyl-1H-pyrazol-3-yl)-N²-[(1R)-2,2,2-trifluoro-1-(4-fluorophenyl)ethyl]pyrimidine-2,4-diamine;
5-Chloro-N⁴-(5-cyclopropyl-1H-pyrazol-3-yl)-N²-[(1S)-2,2,2-trifluoro-1-(4-fluorophenyl)ethyl]pyrimidine-2,4-diamine;
N⁴-(5-Cyclopropyl-1H-pyrazol-3-yl)-N²-[(18)-1-(4-fluorophenyl)ethyl]-7-(2-methoxyethoxy)quinazoline-2,4-diamine;
N⁴-(5-Cyclopropyl-1H-pyrazol-3-yl)-N²-[(1S)-1-(4-fluoro phenyl)ethyl]-7-(2-morpholin-4-ylethoxy)quinazoline-2,4-diamine;
N⁴-(5-Cyclopropyl-1H-pyrazol-3-yl)-N²-[(1S)-1-(4-fluoro phenyl)ethyl]-7-[2-(4-methyl piperazin-1-yl)ethoxy]quinazoline-2,4-diamine;
(2R)-2-{[4-[(5-Cyclopropyl-1H-pyrazol-3-yl)amino]-7-(2-pyrrolidin-1-ylethoxy)quinazolin-2-yl]amino}-2-(4-fluorophenyl)ethanol;
(2R)-2-{[4-[(5-Cyclopropyl-1H-pyrazol-3-yl)amino]-7-(2-morpholin-4-ylethoxy)quinazolin-2-yl]amino}-2-(4-fluorophenyl)ethanol;
N⁴-(5-Cyclopropyl-1H-pyrazol-3-yl)-N²-[(1S)-1-(4-fluorophenyl)ethyl]-7-(2-pyrrolidin-1-ylethoxy)quinazoline-2,4-diamine;
N⁴-(5-Cyclopropyl-1H-pyrazol-3-yl)-N²-[(1S)-1-(4-fluorophenyl)ethyl]-6-(2-pyrrolidin-1-ylethoxy)quinazoline-2,4-diamine;
(2S)-3-[(4-[(5-Cyclopropyl-1H-pyrazol-3-yl)amino]-2-{[(1R)-1-(4-fluorophenyl)-2-hydroxyethyl]amino}quinazolin-7-yl)oxy]propane-1,2-diol;
(2R)-3-[(4-[(5-Cyclopropyl-1H-pyrazol-3-yl)amino]-2-{[(1R)-1-(4-fluorophenyl)-2-hydroxyethyl]amino}quinazolin-7-yl)oxy]propane-1,2-diol;
(2R)-2-({5-Chloro-4-[(5-cyclopropyl-1H-pyrazol-3-yl)amino]-6-[(2-morpholin-4-ylethyl)amino]pyrimidin-2-yl}amino)-2-(4-fluorophenyl)ethanol;
3-[(5-Chloro-6-[(5-cyclopropyl-1H-pyrazol-3-yl)amino]-2-{[(1R)-1-(4-fluorophenyl)-2-hydroxyethyl]amino}pyrimidin-4-yl)amino]propane-1,2-diol;
3-[(5-Chloro-6-[(5-cyclopropyl-1H-pyrazol-3-yl)amino]-2-{[(1R)-1-(4-fluorophenyl)-2-hydroxyethyl]amino}pyrimidin-4-yl)amino]propan-1-ol;
(2R)-2-[(5-Chloro-4-[(5-cyclopropyl-1H-pyrazol-3-yl)amino]-6-{[3-(4-methyl piperazin-1-yl)propyl]amino}pyrimidin-2-yl)amino]-2-(4-fluorophenyl)ethanol;
(2R)-2-({5-Chloro-4-[(5-cyclopropyl-1H-pyrazol-3-yl)amino]-6-[(2-pyrrolidin-1-ylethyl)amino]pyrimidin-2-yl}amino)-2-(4-fluorophenyl)ethanol;
6-[(5-cyclopropyl-1H-pyrazol-3-yl)amino]-2-{[(1R)-1-(4-fluorophenyl)-2-hydroxyethyl]amino}-N-(2-morpholin-4-ylethyl)pyrimidine-4-carboxamide;
(2R)-3-[(6-[(5-Cyclopropyl-1H-pyrazol-3-yl)amino]-2-{[(1S)-1-(4-fluorophenyl)ethyl]amino}pyrimidin-4-yl)amino]propane-1,2-diol;
(2R)-3-({2-{[(1S)-1-(4-Fluorophenyl)ethyl]amino}-6-[(5-methyl-1H-pyrazol-3-yl)amino]pyrimidin-4-yl}amino)propane-1,2-diol;
2-[(6-[(5-Cyclopropyl-1H-pyrazol-3-yl)amino]-2-{[(1S)-1-(4-fluorophenyl)ethyl]amino}pyrimidin-4-yl)amino]ethanol;
2-({2-{[(1S)-1-(4-Fluorophenyl)ethyl]amino}-6-[(5-methyl-1H-pyrazol-3-yl)amino]pyrimidin-4-yl}amino)ethanol;
5-Chloro-N⁴-(5-cyclopropyl-1H-pyrazol-3-yl)-N²-[(1S)-(4-fluoro-phenyl)-ethyl]-pyrimidine-2,4,6-triamine;
5-Chloro-N⁴-(5-cyclopropyl-1H-pyrazol-3-yl)-N²-[(1S)-(4-fluoro-phenyl)-ethyl]-6-(4-methyl-piperazin-1-yl)-pyrimidine-2,4-diamine;
1-Amino-3-[(5-chloro-6-[(5-cyclopropyl-1H-pyrazol-3-yl)amino]-2-{[(1S)-1-(4-fluorophenyl)ethyl]amino}pyrimidin-4-yl)amino]propan-2-ol;
(2R)-2-[(5-Chloro-4-[(5-cyclopropyl-1H-pyrazol-3-yl)amino]-6-{[2-(dimethylamino)ethyl]amino}pyrimidin-2-yl)amino]-2-(4-fluorophenyl)ethanol;
(2R)-2-({5-Chloro-4-[(5-cyclopropyl-1H-pyrazol-3-yl)amino]-6-[(3-pyrrolidin-1-ylpropyl)amino]pyrimidin-2-yl}amino)-2-(4-fluorophenyl)ethanol;
2-[(5-Chloro-6-[(5-cyclopropyl-1H-pyrazol-3-yl)amino]-2-{[(1S)-1-(4-fluorophenyl)ethyl]amino}pyrimidin-4-yl)amino]ethanol;
2-[(5-Chloro-6-[(5-cyclopropyl-1H-pyrazol-3-yl)amino]-2-{[(1S)-1-(4-fluorophenyl)ethyl]amino}pyrimidin-4-yl)amino]propane-1,3-diol;
(2R)-2-{[5-Chloro-4-[(5-cyclopropyl-1H-pyrazol-3-yl)amino]-6-(dimethylamino)pyrimidin-2-yl]amino}-2-(4-fluorophenyl)ethanol;
1-Amino-3-[(5-chloro-6-[(5-cyclopropyl-1H-pyrazol-3-yl)amino]-2-{[(1R)-1-(4-fluorophenyl)-2-hydroxyethyl]amino}pyrimidin-4-yl)amino]propan-2-ol;
2-[(5-Chloro-6-[(5-cyclopropyl-1H-pyrazol-3-yl)amino]-2-{[(1R)-1-(4-fluorophenyl)-2-hydroxyethyl]amino}pyrimidin-4-yl)amino]propane-1,3-diol;
(2R)-2-[(5-Chloro-4-[(5-cyclopropyl-1H-pyrazol-3-yl)amino]-6-{[2-(2-hydroxy ethoxy)ethyl]amino}pyrimidin-2-yl)amino]-2-(4-fluorophenyl)ethanol;
(2R)-3-[(5-Chloro-6-[(5-cyclopropyl-1H-pyrazol-3-yl)amino]-2-{[(1S)-1-(4-fluoro phenyl)ethyl]amino}pyrimidin-4-yl)amino]propane-1,2-diol;
(2R)-2-{[5-Chloro-4-[(5-cyclopropyl-1H-pyrazol-3-yl)amino]-6-(ethylamino) pyrimidin-2-yl]amino}-2-(4-fluorophenyl)ethanol;
(2S)-3-[(5-Chloro-6-[(5-cyclopropyl-1H-pyrazol-3-yl)amino]-2-{[(1R)-1-(4-fluorophenyl)-2-hydroxyethyl]amino}pyrimidin-4-yl)amino]propane-1,2-diol;
(2R)-3-[(5-Chloro-6-[(5-cyclopropyl-1H-pyrazol-3-yl)amino]-2-{[(1R)-1-(4-fluorophenyl)-2-hydroxyethyl]amino}pyrimidin-4-yl)amino]propane-1,2-diol;
(2R)-2-({5-Chloro-4-[(5-cyclopropyl-1H-pyrazol-3-yl)amino]-6-[(2-hydroxyethyl)amino]pyrimidin-2-yl}amino)-2-(4-fluorophenyl)ethanol;
(2R)-2-{[5-Chloro-4-[(5-cyclopropyl-1H-pyrazol-3-yl)amino]-6-(methylamino)pyrimidin-2-yl]amino}-2-(4-fluorophenyl)ethanol;
(2S)-1-[(5-Chloro-6-[(5-cyclopropyl-1H-pyrazol-3-yl)amino]-2-{[(1S)-1-(4-fluorophenyl)ethyl]amino}pyrimidin-4-yl)amino]propan-2-ol;
3-[(5-Chloro-6-[(5-cyclopropyl-1H-pyrazol-3-yl)amino]-2-{[(1S)-1-(4-fluorophenyl)ethyl]amino}pyrimidin-4-yl)amino]-1,1,1-trifluoropropan-2-ol;
3-[(5-Chloro-6-[(5-cyclopropyl-1H-pyrazol-3-yl)amino]-2-{[(1S)-1-(4-fluorophenyl)ethyl]amino}pyrimidin-4-yl)(methyl)amino]propane-1,2-diol;
5-Chloro-N⁴-(5-cyclopropyl-1H-pyrazol-3-yl)-N²-[(1S)-1-(4-fluorophenyl)ethyl]-6-morpholin-4-ylpyrimidine-2,4-diamine;
(2R)-2-({5-Chloro-4-[(5-cyclopropyl-1H-pyrazol-3-yl)amino]-6-morpholin-4-ylpyrimidin-2-yl}amino)-2-(4-fluorophenyl)ethanol;
(2R)-2-{[5-Chloro-4-[(5-cyclopropyl-1H-pyrazol-3-yl)amino]-6-(4-methylpiperazin-1-yl)pyrimidin-2-yl]amino}-2-(4-fluorophenyl)ethanol;
5-Chloro-N⁴-(5-cyclopropyl-1H-pyrazol-3-yl)-N²-[(1S)-1-(4-fluorophenyl)ethyl]-6-pyrrolidin-1-ylpyrimidine-2,4-diamine;
(2R)-3-[(5-Chloro-6-[(3-ethoxy-1H-pyrazol-5-yl)amino]-2-{[(1S)-1-(4-fluorophenyl)ethyl]amino}pyrimidin-4-yl)amino]propane-1,2-diol;
(2R)-3-({5-Chloro-2-{[(1S)-1-(4-fluorophenyl)ethyl]amino}-6-[(3-isopropoxy-1H-pyrazol-5-yl)amino]pyrimidin-4-yl}amino)propane-1,2-diol;
(2R)-3-({5-Chloro-2-{[(1R)-1-(4-fluorophenyl)-2-hydroxyethyl]amino}-6-[(3-isopropoxy-1H-pyrazol-5-yl)amino]pyrimidin-4-yl}amino)propane-1,2-diol;
2-({5-Chloro-2-{[(1S)-1-(4-fluorophenyl)ethyl]amino}-6-[(5-isopropoxy-1H-pyrazol-3-yl)amino]pyrimidin-4-yl}amino)propane-1,3-diol;
2-({5-Chloro-2-{[(1R)-1-(4-fluorophenyl)-2-hydroxyethyl]amino}-6-[(5-isopropoxy-1H-pyrazol-3-yl)amino]pyrimidin-4-yl}amino)propane-1,3-diol;
N⁴-(5-Cyclopropyl-2H-pyrazol-3-yl)-N²-[(S)-1-(4-fluorophenyl)-ethyl]-5,6,7,8-tetrahydro-pyrido[4,3-d]pyrimidine-2,4-diamine;
N⁴-(5-Cyclopropyl-1H-pyrazol-3-yl)-N²-[(S)-1-(4-fluoro-phenyl)-ethyl]-5,6,7,8-tetrahydro-pyrido[2,3-a]pyrimidine-2,4-diamine;
N⁴-(5-Cyclopropyl-1H-pyrazol-3-yl)-N²-[(S)-1-(4-fluoro-phenyl)-ethyl]-5,6,7,8-tetrahydro-pyrido[3,4-d]pyrimidine-2,4-diamine;
1-{4-(5-Cyclopropyl-1H-pyrazol-3-ylamino)-2-[(S)-1-(4-fluoro-phenyl)-ethylamino]-7,8-dihydro-5H-pyrido[4,3-d]pyrimidin-6-yl}-ethanone;
1-{4-(5-Cyclopropyl-1H-pyrazol-3-ylamino)-2-[(8)-1-(4-fluoro-phenyl)-ethylamino]-5,8-dihydro-6H-pyrido[3,4-d]pyrimidin-7-yl}-ethanone;
5-Chloro-N⁴-(5-cyclopropyl-1H-pyrazol-3-yl)-N²-[2,2,2-trifluoro-1-(4-fluorophenyl)ethyl]pyrimidine-2,4-diamine;
4-(5-Cyclopropyl-1H-pyrazol-3-ylamino)-2-[(S)-1-(4-fluoro-phenyl)-ethylamino]-7,8-dihydro-5H-pyrido[4,3-d]pyrimidine-6-carboxylic acid benzyl ester;
4-(5-Cyclopropyl-1H-pyrazol-3-ylamino)-2-[(S)-1-(4-fluoro-phenyl)-ethylamino]-5,8-dihydro-6H-pyrido[3,4-d]pyrimidine-7-carboxylic acid benzyl ester;
6-Chloro-N²-[(1S)-1-(4-fluorophenyl)ethyl]-N-(5-methyl-1H-pyrazol-3-yl)pyrimidine-2,4-diamine;
5,6-Dichloro-N⁴-(5-ethoxy-1H-pyrazol-3-yl)-N²-[(1S)-1-(4-fluorophenyl)ethyl]pyrimidine-2,4-diamine;
5,6-Dichloro-N²-[(1S)-1-(4-fluorophenyl)ethyl]-N⁴-(3-isopropoxy-1H-pyrazol-5-yl)pyrimidine-2,4-diamine;
(2R)-2-({4,5-Dichloro-6-[(3-isopropoxy-1H-pyrazol-5-yl)amino]pyrimidin-2-yl}amino)-2-(4-fluorophenyl)ethanol;
5-bromo-N⁴-(5-methyl-1H-pyrazol-3-yl)-N²-[1-(2-pyridinyl)propyl]-2,4-pyrimidinediamine;
5-chloro-N⁴-(5-methyl-1H-pyrazol-3-yl)-N²-[1-(2-pyridinyl)propyl]-2,4-pyrimidinediamine;
5-bromo-N²-[1-(3-methyl-5-isoxazolyl)ethyl]-N⁴-(5-methyl-1H-pyrazol-3-yl)-2,4-pyrimidinediamine;
5-chloro-N²-[1-(3-methyl-5-isoxazolyl)ethyl]-N⁴-(5-methyl-1H-pyrazol-3-yl)-2,4-pyrimidinediamine;
5-bromo-N⁴-(5-methyl-1H-pyrazol-3-yl)-N²-[1-(3-pyridinyl)propyl]-2,4-pyrimidinediamine;
5-chloro-N⁴-(5-methyl-1H-pyrazol-3-yl)-N²-[1-(3-pyridinyl)propyl]-2,4-pyrimidinediamine;
5-chloro-N⁴-(5-methyl-1H-pyrazol-3-yl)-N²-[1-(3-pyridinyl)ethyl]-2,4-pyrimidinediamine;
5-bromo-N⁴-(5-methyl-1H-pyrazol-3-yl)-N²-[1-(3-pyridinyl)ethyl]-2,4-pyrimidinediamine; or
5-bromo-N⁴-(5-methyl-1H-pyrazol-3-yl)-N²-[1-(2-pyridinyl)ethyl]-2,4-pyrimidinediamine.
   or a pharmaceutically acceptable salt thereof.

A suitable process for preparing a compound of formula (I) or a pharmaceutically acceptable salt thereof which process (wherein variable groups are, unless otherwise specified, as defined in formula (I)) comprises of:

Process a) reaction of a pyrimidine of formula (II):

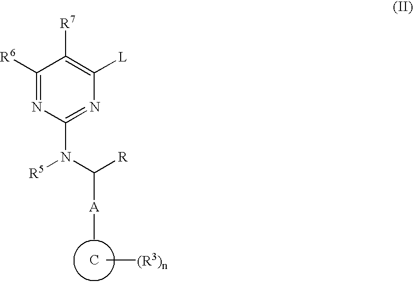

wherein L is a displaceable group; with an pyrazole amine of formula (III):

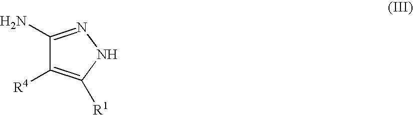

or

Process b) reacting a pyrimidine of formula (IV):

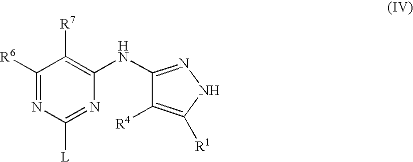

wherein L is a displaceable group; with a compound of formula (V):

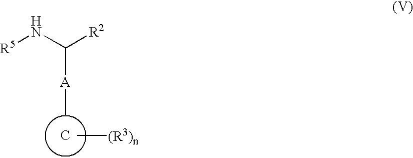

Process c) reacting a compound of formula (VI):

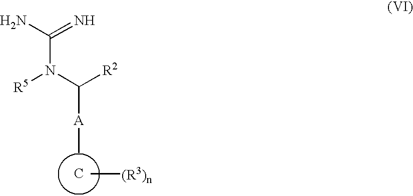

with a compound of formula (VII):

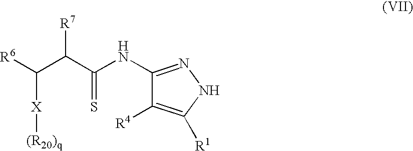

wherein X is an oxygen atom and q is 1; or X is a nitrogen atom and q is 2; and wherein each R²⁰ independently represents a C₁₋₆alkyl group; or

Process d) reacting a compound of formula (VIII):

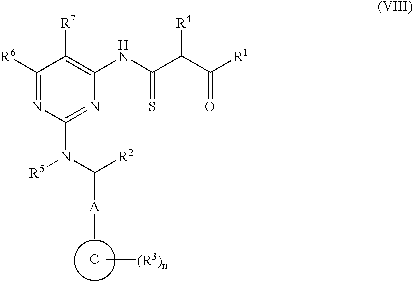

with hydrazine; or
and thereafter if necessary:
i) converting a compound of the formula (a) into another compound of the formula (I);
ii) removing any protecting groups;
iii) forming a pharmaceutically acceptable salt.

L is a displaceable group, suitable values for L are for example, a halo or sulphonyloxy group, for example a chloro, bromo, methanesulphonyloxy or toluene-4-sulphonyloxy group.

Specific reaction conditions for the above reactions are as follows.

Process a) Pyrimidines of formula (II) and pyrazole amine of formula (III) may be reacted together:

a) in the presence of a suitable solvent for example a ketone such as acetone or an alcohol such as ethanol or butanol or an aromatic hydrocarbon such as toluene or N-methyl pyrrolid-2-one, optionally in the presence of a suitable acid for example an inorganic acid such as hydrochloric acid or sulphuric acid, or an organic acid such as acetic acid or formic acid (or a suitable Lewis acid) and at a temperature in the range from 0° C. to reflux, particularly reflux; or
b) under standard Buchwald conditions (for example see J. Am. Chem. Soc., 118, 7215; J. Am. Chem. Soc., 119, 8451; J. Org. Chem., 62, 1568 and 6066) for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl and at a temperature in the range from 25 to 80° C.

Pyrimidines of the formula (II) may be prepared according to Scheme 1:

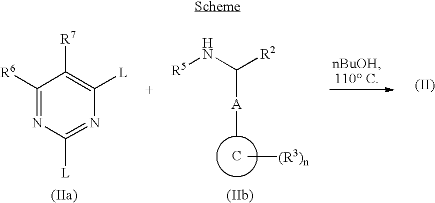

Pyrazole amines of formula (III) and compound of formula (IIa) and (Ib) are commercially available compounds, or they are known in the literature, or they are is prepared by standard processes known in the art.

Process b) Compounds of formula (IV) and formula (V) may be reacted together under the same conditions as outlined in Process a).

Compounds of the formula (IV) may be prepared according to Scheme 2:

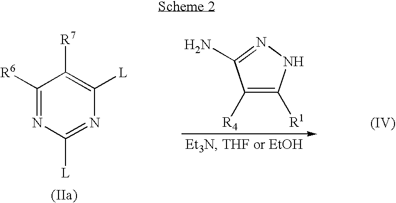

Compounds of the formula (V) are commercially available compounds, or they are known in the literature, or they are prepared by standard processes known in the art.

Process c) may conveniently be carried out in a suitable solvent such as N-methylpyrrolidinone or butanol at a temperature in the range from 100-200° C., in particular in the range from 150-170° C. The reaction is preferably conducted in the presence of a suitable base such as, for example, sodium methoxide or potassium carbonate.

Compounds of the formula (VI) may be prepared according to Scheme 3:

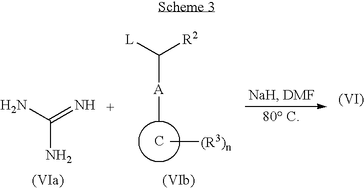

Compounds of the formula (VII) may be prepared according to Scheme 4:

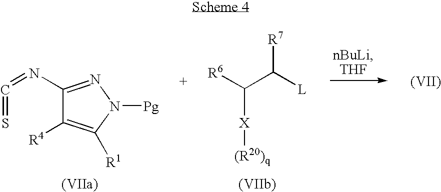

wherein Pg is a suitable nitrogen-protecting group. Suitable values for Pg are defined below.

Compounds of the formula (VIa), (VIb), (VIIa) and (VIb) are commercially available compounds, or they are known in the literature, or they are prepared by standard processes known in the art.

Process d) may be carried out in a suitable solvent, for example, an alcohol such as ethanol or butanol at a temperature in the range from 50-120° C., in particular in the range from 70-100° C.

Compounds of the formula (VIII) may be prepared according to Scheme 5:

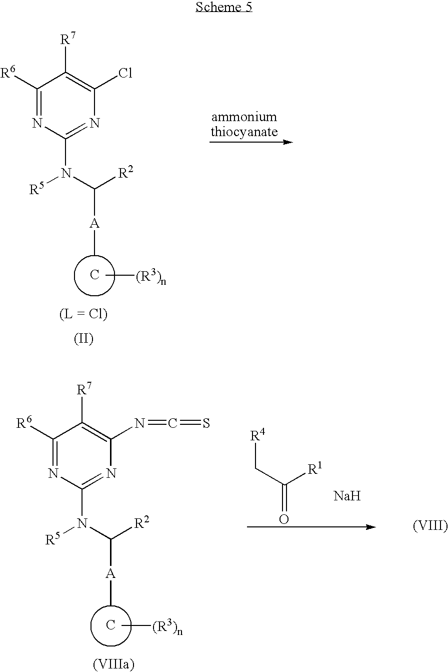

It will be appreciated that certain of the various ring substituents in the compounds to be used according to the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

### DEFINITIONS

In this specification the term “alkyl” includes both straight and branched chain alkyl groups but references to individual alkyl groups such as “propyl” are specific for the straight chain version only. For example, “C₁₋₆alkyl” and “C₁₋₄alkyl” include methyl, ethyl, propyl, isopropyl and t-butyl. However, references to individual alkyl groups such as ‘propyl’ are specific for the straight-chained version only and references to individual branched chain alkyl groups such as ‘isopropyl’ are specific for the branched-chain version only. A similar convention applies to other radicals. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, and a ring sulphur atom may be optionally oxidised to form the S-oxides. Particularly “heterocyclyl” is a heteroaryl, such as aza-, thia-, oxa-, oxaza-, thiaza- or diazacycloalkyl, aza-, thia-, oxa-, oxaza-, thiaza- or diazacycloalkenyl, azaaryl, thiazaaryl or oxazaaryl. Examples and suitable values of the term “heterocyclyl” are morpholino, piperidyl, pyridyl, pyranyl, pyrrolyl, furyl, isothiazolyl, indolyl, quinolyl, thienyl, 1,3-benzodioxolyl, thiadiazolyl, piperazinyl, thiazolidinyl, pyrrolidinyl, thiomorpholino, piperazinyl, pyrrolinyl, homopiperazinyl, 3,5-dioxapiperidinyl, tetrahydropyranyl, imidazolyl, pyrimidyl, pyrazinyl, pyridazinyl, isoxazolyl, oxazolyl, N-methylpyrrolyl, 4-pyridone, 1-isoquinolone, 2-pyrrolidone, 4-thiazolidone, pyridine-N-oxide and quinoline-N-oxide.

A “carbocyclyl” is a saturated, partially saturated or unsaturated, mono or bicyclic carbon ring that contains 3-12 atoms, such as cycloalkyl, cycloalkenyl or aryl; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

Where “R⁶ and R⁷ together with the bond to which they are attached form a 5 or 6 membered heterocyclic ring” said ring is a partially saturated or unsaturated, mono or bicyclic carbon ring that contains 5 or 6 atoms two atoms of which are shared with the pyrimidine ring of formula (I); of which at least one atom is chosen from nitrogen, sulphur or oxygen; wherein a —CH₂— group can optionally be replaced by a —C(O)—, and a ring sulphur atom may be optionally oxidized to form the S-oxides. Said ring is fused to the pyrimidine ring of formula (I) to make a 9 or 10 membered bicyclic ring. Suitable values for “R⁶ and R⁷ together with the bond to which they are attached form a 5 or 6 membered heterocyclic ring wherein said ring is fused to the pyrimidine ring in formula (I)” are pteridinyl, purinyl, thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl. Further suitable values for “R⁶ and R⁷ together with the bond to which they are attached form a 5 or 6 membered heterocyclic ring wherein said ring is fused to the pyrimidine ring in formula (I)” are thieno[3,2-d]pyrimidinyl, thieno[2,3-d]pyrimidinyl, thieno[3,4-d]pyrimidinyl, 1H-pyrazolo[3,4-d]pyrimidinyl or pyrido[2,3-d]pyrimidinyl. Additional suitable values for “R⁶ and R⁷ together with the bond to which they are attached form a 5 or 6 membered heterocyclic ring wherein said ring is fused to the pyrimidine ring in formula (I)” are thieno[3,2-d]pyrimidinyl, thieno[2,3-d]pyrimidinyl, 1H-pyrazolo[3,4-d]pyrimidinyl, thieno[3,4-d]pyrimidinyl, pyrido[2,3-d]pyrimidinyl, 5,6,7,8-tetrahydro-pyrido[4,3-d]pyrimidinyl, 5,6,7,8-tetrahydro-pyrido[2,3-d]pyrimidinyl and 5,6,7,8-tetrahydro-pyrido[3,4-d]pyrimidinyl.

Where “R⁶ and R⁷ together with the bond to which they are attached form a 5 or 6 membered carbocyclic ring” said ring is a partially saturated or unsaturated, mono or bicyclic carbon ring that contains 5 or 6 atoms two atoms of which are shared with the pyrimidine ring of formula (I); wherein a —CH₂— group can optionally be replaced by a —C(O)—. Said ring is fused to the pyrimidine ring of formula (I) to make a 9 or 10 membered bicyclic ring. Suitable values for “R⁶ and R⁷ together with the bond to which they are attached form a 5 or 6 membered carbocyclic ring wherein said ring is fused to the pyrimidine ring in formula (I)” are quinazolinyl.

The term “Cₘ₋ₙ” or “Cₘ₋ₙ group” used alone or as a prefix, refers to any group having m to n carbon atoms.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

For compounds of formula (I) additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, is xanthene, phenoxathiin, thianthrene, indolizine, isoindole, indazole, purine, phthalazine, naphthyridine, quinoxaline, quinazoline, cirmoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

For compounds of formula (I) in addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

For compounds of formula (I) heterocyclyl includes, for example, monocyclic heterocyclyls, such as: aziridinyl, oxiranyl, thuiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

For compounds of formula (I) in addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

For compounds of formula (I) additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

For compounds of formula (I) in addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

For compounds of formula (I) the term “amine” or “amino” used alone or as a suffix or prefix, refers to radicals of the general formula —NRR′, wherein R and R′ are independently selected from hydrogen or a hydrocarbon radical.

An example of “C₁₋₆alkanoyloxy” is acetoxy. Examples of “C₁₋₆alkoxycarbonyl” include C₁₋₄alkoxycarbonyl, methoxycarbonyl, ethoxycarbonyl, n- and t-butoxycarbonyl. Examples of “C₁₋₆alkoxy” include C₁₋₄alkoxy, C₁₋₃alkoxy, methoxy, ethoxy and propoxy. Examples of “C₁₋₆alkoxyimino” include C₁₋₄alkoxyimino, C₁₋₃alkoxyimino, methoxyimino, ethoxyimino and propoxyimino. Examples of “C₁₋₆alkanoylamino” include formamido, acetamido and propionylamino. Examples of “C₁₋₆alkylS(O)ₐ wherein a is 0 to 2” include C₁₋₄alkylsulphonyl, methylthio, ethylthio, methylsulphinyl, ethylsulphinyl, mesyl and ethylsulphonyl. Examples of “CC₁₋₆alkylthio” include methylthio and ethylthio. Examples of “C₁₋₆alkylsulphonylamino” include methylsulphonylamino and ethylsulphsulphonylamino. Examples of “C₁₋₆alkanoyl” include C₁₋₄alkanoyl, propionyl and acetyl. Examples of “N—(C₁₋₆alkyl)amino” include methylamino and ethylamino. Examples of “N,N—(C₁₋₆alkyl)₂-amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂-carbamoyl” are N,N—(C₁₋₄alkyl)₂-carbamoyl, dimethylaminocarbonyl and methylethylaminocarbonyl.

A first ring group being “fused” with a second ring group means the first ring and the second ring share at least two atoms there between.

A suitable pharmaceutically acceptable salt of a compound to be used according to the invention is, for example, an acid-addition salt of a compound to be used according to the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound to be used according to the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

It should be noted that the pyrazolyl-pyrimidines used according to this invention are capable of existing as different stereoisomeric and tautomeric structures and thus the pyrazolyl-pyrimidines claimed herein include all these possibilities, for example optical isomers, diastereoisomers and geometric isomers and all tautomeric forms of the compounds of the formula (I).

It is also to be understood that certain compounds of the formula (I) can exist in solvated as well as unsolvated forms such as, for example, hydrated forms. It is to be understood that the invention encompasses all such solvated forms.

The wording “daily dose” is defined so that the pyrazolyl-pyrimidines compounds may be given either as a unit dosage once daily, such as a tablet or a capsule, or alternatively the pyrazolyl-pyrimidines compounds may be given twice daily. The daily dose may vary within the dosage ranges mentioned below, and depends on the patient's individual response to treatment.

With the wording “therapeutic treatment” as herein used, is meant that pain is treated by administering a pyrazolyl-pyrimidine derivative according to the formula I above, as soon as the pain has started to give the patient suffering therefrom, to relieve pain sensations. This means that the use of a pyrazolyl-pyrimidine derivative according to the formula I above, provides therapy of a fully or partly developed pain condition such as pain caused by chemical, mechanical, radiation, thermal, infectious or inflammatory tissue trauma or cancer.

With the wording “prophylactic treatment” as herein used, is meant that a pyrazolyl-pyrimidine derivative according to the formula I above, may be administered to a person to prevent the frequency of pain attacks and to reduce the severity or the duration of the attack. Furthermore, it may be administered before the pain attack has started to give fall symptoms or only slight symptoms.

### Usage

In accordance with the present invention, the applicant has hereby found that the compounds of formula (I) which possess kinase inhibitory activity are useful for the treatment or prophylaxis of pain conditions and are therefore useful in methods of treatment of human or animal body. The invention also relates to pharmaceutical formulations containing said pyrazolyl-pyrimidines compounds and to their use in the manufacture of medicaments of use with the production of analgesic effect in warm-blooded animals such as man.

The present invention includes use of pharmaceutically acceptable salts or pro-drugs of such compounds. Also in accordance with the present invention applicants provide pharmaceutical formulations and a method to use such compounds in the treatment of pain.

The properties of the compounds used according to the claimed invention are expected to be useful in therapy of value in the treatment of pain states especially for the treatment and/or prophylaxis of pain which may be of widely different origins and causes and include acute as well as chronic pain states. Examples are pain caused by chemical, mechanical, radiation, thermal, infectious or inflammatory tissue trauma or cancer. Additional examples are posttraumatic pain, headache and migraine, various arthritic and inflammatory conditions such as osteo and rheumatoid arthritis, myofascial and low back pain associated with chronic inflammation, bone diseases, and cell proliferation such as cancers (solid tumors and leukemia).

Also neuropathic conditions of central or peripheral origin can be treated or prevented according to the invention. Examples of these pain conditions are trigeminal neuralgia, postherpetic neuralgia (PHN), painful diabetic mono/poly neuropathy, and pain associated with nerve damage, spinal cord injury, central post stroke, multiple sclerosis and Parlinson's disease.

Other pain states of visceral origin such as caused by ulcer, dysmenorrhea, endometriosis, IBS, dyspepsia etc. can also be treated or prevented with the compounds of the formula (I).

A primary aim of the invention is to use compounds of the formula (J) for oral treatment of chronic inflammatory or neuropathic pain states.

The typical daily dose of the active substance necessarily varies within a wide range and will depend on various factors such as for example the individual requirement of each patient, the route of administration and the disease. In general, the dosages will be in the range of 1 to 1000 mg per day of active substance.

### Formulations

Compounds used according to the present invention may be administered orally or parenterally and can be administered by the buccal, vaginal, rectal, inhalation, insufflation, sublingual, intramuscular, subcutaneous, topical, intranasal, intraperitoneal, intrathoracial, intravenous, epidural, intrathecal, intracerebroventricular routes and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound used according to the present invention for use in therapy of pain is an amount sufficient to symptomatically relieve in a warm-blooded animal, particularly a human the sensations of pain, to slow the progression of pain sensations, or to reduce in patients with pain the risk of experiencing worse pain.

For preparing pharmaceutical formulations from the compounds used according to this invention, inert, pharmaceutically acceptable carriers can be either solid or liquid. Solid form preparations include powders, tablets, dispersible granules, capsules, cachets, and suppositories.

A solid carrier can be one or more substance, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository formulations, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds used according to the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminium, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical formulation.

In addition to the compounds used according to the present invention, the pharmaceutical formulation of this invention may also contain, or be co-administered (simultaneously or sequentially) with, one or more pharmacological agents of value in treating one or more disease conditions referred to herein.

The term formulation is intended to include the formulation of the active component or a pharmaceutically acceptable salt with a pharmaceutically acceptable carrier. For example this formulation may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form formulations include solutions, suspensions, and emulsions. Sterile water or water-propylene glycol solutions of the active compounds may be mentioned as an example of liquid preparations suitable for parenteral administration. Liquid formulations can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known to the pharmaceutical formulation art.

The pharmaceutical formulations can be in unit dosage form. In such form, the formulation is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

### Combinations

The pain treatment defined herein may be applied as a sole therapy or may involve, in addition to the compound used according to the invention, administration of other analgesics or adjuvant therapy. Such therapy may for example include, in combination for simultaneous, separate or sequential use one or more of the following categories of pain-relieving ingredients
a) opioid analgesics, for example morphine, ketobemidone or fentanyl
b) analgesics of the NSAID or COX-1 or COX-2 class, for example ibuprofen, naproxen, celecoxib or acetylsalicylic acid, and their analogues containing nitric oxide-donating groups.
c) analgesic adjuvants such as amitriptyline, imipramine, duloxetine or mexiletine
d) NMDA antagonists for example ketamine, memantine or dextrometorfan
e) sodium channel blocking agents, for example lidocaine or mexiletine
f) anticonvulsants, for example carbamazepine, topiramate or lamotrigine
g) anticonvulsant/analgesic amino acids such as gabapentin or pregabalin
h) cannabinoids
i) antibodies directed towards NGF or TNF-alpha

### Biological Tests

### In Vivo Experiments

The compounds used according to the invention when given by systemic administration to mice or rats, specifically reduce pain in the rat carrageenan test as described by Tonussi and Ferreira (Pain 1992, 48, 421-427).

It can therefore be inferred that the compounds can be used as therapeutic agents to relieve pain of various origins. The compounds used according to the invention exhibit effective doses by oral or subcutaneous administration to rats in the range from about 10 to about 80 mg/kg.

The analgesic activity of the compounds can also be assessed by several other methods, for example by mouse or rat behavior in the formalin test. This test is an accepted model of clinical pain in man, involving elements of nociceptor activation, inflammation, peripheral sensitization and central sensitization (A Tjølsen et al. Pain 1992, 51, 5). The analgesic activity of compounds of formula I can also be shown in the intraarticular FCA (Freund's complete adjuvant) test in the rat, a model of inflammatory pain (Iadarola et al. Brain Research 1988, 455, 205-12) and in the Chung nerve lesion test in the rat, a model for neuropathic pain (Kim and Chung. Pain 1992, 50, 355).

### Description of the Rat Carrageenan Test

Experimental procedures. Under isoflurane anesthesia, 40 μL of carrageenan (7.5 mg/mL) was injected into the left tibio-tarsal (ankle) joint from the dorsal side. The injection causes a localized inflammation increasing to a maximum between 4 h to 6 h after induction, after which it gradually decreases, and the animals display decreased weight bearing on and guarding of the limb.

(2R)-2-{[5-Chloro-4-(1H-pyrazol-5-ylamino)pyrimidin-2-yl]amino}-2-(4-fluorophenyl)ethanol was administered 30 min before induction of monoarthritis. The rats were placed in an acrylic chamber and videotaped for 5 min from underneath, 2 h, 2 h30, 3 h, 3 h30, 5 h after drug administration. Subsequently, the weight the rats were willing to put on the injected paw was scored as described in the table below, and defined as the “Pain Score”:

| | |
|---|---|
| 0 | normal paw position |
| 0.5 | normal paw position, but slightly reduced spread of the toes |
| 1 | the paw is used during walking, but the toes are kept together |
| 1.5 | the toes are kept together, and the animal is sometimes limping |
| 2 | pronounced limping |
| 2.5 | the paw only occasionally touches the floor |
| 3 | the paw does not contact the floor |

Data analysis. Two Way Repeated Measures ANOVA (one factor repetition) followed by Newmans-Keuls method for all pairwise multiple comparison procedures (SigmaStat® 2.03) was used to evaluate group effects. The level of significance was set at p<0.05.

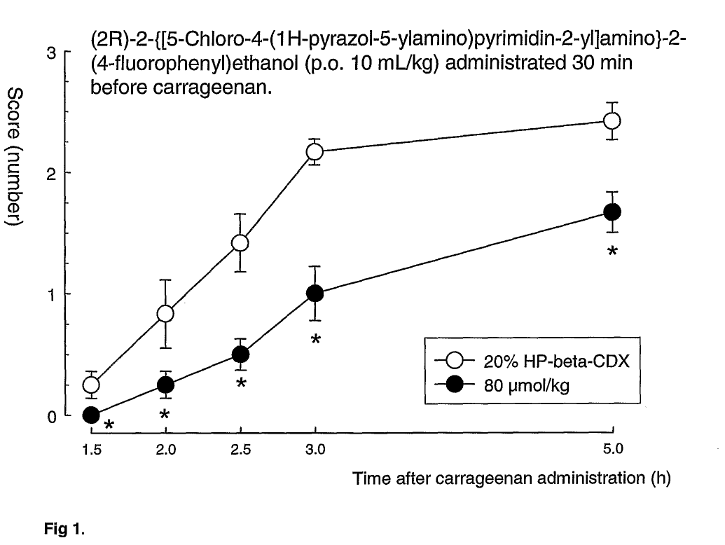

Result. Oral administration of (2R)-2-{[5-Chloro-4-(1H-pyrazol-5-ylamino)pyrimidin-2-yl]amino}-2-(4-fluorophenyl)ethanol at a dose of 80 μmol/kg significantly reduced the pain score induced by carrageenan monoarthritis within 5 hours after administration (FIG. 1). The animals did not show signs of side-effects.

### TrkB Assay Format

TrkB kinase activity is being measured against its ability to phosphorylate synthetic tyrosine residues within a generic polypeptide substrate using homogenous time-resolved fluorescence (HTRF) technology. The intracellular domain of a HIS-tagged human TrkB kinase was expressed in SF9 cells and purified using standard nickel column chromatography. After the kinase is incubated with a biotinylated substrate and ATP for 50 minutes at room temperature, the kinase reaction is stopped by the addition of 60 mM EDTA. The reaction is performed in 384 well microtitre plates and reaction products are detected with the addition of strepavidin-linked and phosphotyrosine-specific antibodies using the Tecan Ultra Evolution Microplate Fluometer after an additional 3-hour incubation at room temperature.

| | |
|---|---|
| Peptide substrate | PolyEAY-biotin (PGAT-bio.) |
| ATP Km | 60 uM |
| Assay conditions | 400 ng/ml TrkB, 10 mM HEPES, 0.005% BR SA, |
| | 20 mM MnCl₂, 100 nM PGAT-bio, 120 nM ATP |
| Incubation | 50 minutes, room temperature |
| Termination/ | 50 mM HEPES, 60 mM EDTA, 0.03% BR SA, |
| Detection conditions | 5.9 nM p-Tyr LANCE Ab, 45 nM XL-665 Ab |
| Detection incubation | 3 hours, room temperature |
| Fluometer settings | Excitation = 340 nM |
| | Emission 1 = 612 nM |
| | Emission 2 = 670 nM |
| | Flash = 10 |
| | Integration = 200 us |
| | Lad = 50 us |

TrkA Assay Format

TrkA kinase activity is measured in 384-well format using AlphaScreen technology. The synthetic substrate used is a biotin-conjugated poly-glutamate, tyrosine (4:1) peptide (PGT), which has been shown to be a specific substrate for tyrosine kinases. The intracellular domain of a His-tagged human TrkA kinase was expressed in High Five cells. The kinase was incubated with ATP and the biotinylated substrate for 15 minutes at room temperature. The reactions were then stopped by the addition of EDTA, anti-phosphotyrosine antibody (PT-100) coated acceptor beads and streptavidin coated donor beads. After 2 h of incubation in the dark at room temperature, the product was detected using a Fusion Alpha instrument. Assay conditions: 1.76 nM TrkA, 20 μM ATP (=Km for ATP), 0.5 μg/ml PGT, 13 mM MnCl₂, 40 mM Tris pH 7.4, 100 mM NaCl and 0.075% BSA. Termination/Detection conditions: 25 mM HEPES pH 7.4, 100 mM NaCl, 48 mM EDTA, 0.1% BSA and 20 μg/ml of donor and acceptor beads.

When tested in the above in-vitro assay the Trk inhibitory activity of the following examples was measured at the following IC₅₀s.

| Compound | TrkA IC₅₀ (μM) | TrkB IC₅₀ (μM) |
|---|---|---|
| 5-Chloro-N⁴-(5-cyclopropyl-1H- | 0.035 | 0.14 |
| pyrazol-3-yl)-N²-[(1S)-1-(4- | | |
| fluorophenyl)ethyl]pyrimidine-2,4- | | |
| diamine | | |
| (3S)-3-({4-[(5-Cyclopropyl-1H- | 0.039 | 0.22 |
| pyrazol-3-yl)amino]-5- | | |
| chloropyrimidin-2-yl}amino)-3-(4- | | |
| fluorophenyl)propan-1-ol | | |
| (2R)-2-({4-[(5-Cyclopropyl-1H- | 0.075 | 0.26 |
| pyrazol-3-yl)amino]-5- | | |
| fluoropyrimidin-2-yl}amino)-2-(4- | | |
| fluorophenyl)ethanol | | |

1. A method of treating or preventing pain, the method comprising administering to a subject a compound of formula (I): 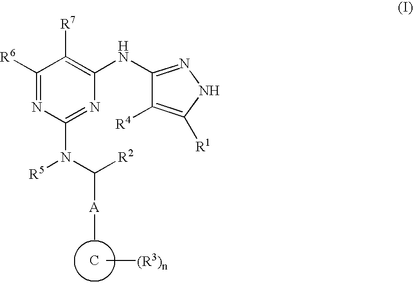
wherein:
A is a, direct bond or C₁₋₂alkylene; wherein said C₁₋₂alkylene may be optionally substituted by one or more R²²;
Ring C is carbocyclyl or heterocyclyl;
R¹ and R⁴ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹ and R⁴ independently of each other may be optionally substituted on carbon by one or more R⁸; and wherein if said heterocyclyl contains an —NH— moiety that nitrogen may be optionally substituted by a group selected from R⁹;
R² is selected from hydrogen, cyano, carbamoyl, sulphamoyl, C₁₋₆alkyl, C₂₋₆alkenyl, C₂₋₆alkynyl, C₁₋₆alkoxy, C₁₋₆alkanoyl, N—(C₁₋₆alkyl)carbamoyl, N,N—(C₁₋₆alkyl)₂carbamoyl, C₁₋₆alkylS(O)ₐ, wherein a is 0 to 2, C₁₋₆alkoxycarbonyl, N—(C₁₋₆alkyl)sulphamoyl, N,N—(C₁₋₆alkyl)₂sulphamoyl, carbocyclyl or heterocyclyl; wherein R² may be optionally substituted on carbon by one or more R¹⁰— and wherein if said heterocyclyl contains an —NH— moiety that nitrogen may be optionally substituted by a group selected from R¹¹;
R³ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R³ may be optionally substituted on carbon by one or more R¹²; and wherein if said heterocyclyl contains an —NH— moiety that nitrogen may be optionally substituted by a group selected from R¹³;
R⁵ is hydrogen or optionally substituted C₁₋₆alkyl; wherein said optional substituents are selected from one or more R¹⁴;
R⁶ and R⁷ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆-alkyl)₂amino, C₁₋₆alkanoylamino, N—(C₁₋₆alkyl)carbamoyl, N,N—(C₁₋₆alkyl)₂carbamoyl, C₁₋₆,alkylS(O)ₐ wherein a is 0 to 2, C₁₋₆alkoxycarbonyl, N,N—(C₁₋₆alkyl)sulphamoyl, N,N—(C₆alkyl)₂sulphamoyl, C₁₋₆alkylsulphonylamino, carbocyclyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶;
or R⁶ and R⁷ together with the bond to which they are attached form a 5 or 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring, may be optionally substituted on carbon by one or more R¹⁷; and wherein if said heterocyclic ring contains an —NH— moiety that nitrogen may be optionally substituted by a group selected from R¹⁸;
n=0, 1, 2 or 3; wherein the values of R³ may be the same or different;
R⁸, R¹⁰, R¹², R¹⁴, R¹⁵, R¹⁷ and R²² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ, wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁸, R¹⁰, R¹², R¹⁴, R¹⁵, R¹⁷ and R²² independently of each other may be optionally substituted on carbon by one or more R¹⁹; and wherein if said heterocyclyl contains an —NH— moiety that nitrogen may be optionally substituted by a group selected from R²⁰;
R⁹, R¹¹, R¹³, R¹⁶, R¹⁸ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹¹, R¹³, R¹⁶, R¹⁸ and R²⁰ independently of each other may be optionally substituted on carbon by on or more R²¹;
R¹⁹ and R²¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂carbamoyl, C₁₋₆alkylS(O)ₐ, wherein a is 0 to 2, C₁₋₆alkoxycarbonyl, N—(C₁₋₆alkyl)sulphamoyl, N,N—(C)₆alkyl)₂sulphamoyl, C₁₋₆alkylsulphonylamino, carbocyclyl or heterocyclyl; wherein R¹⁹ and R²¹ independently of each other may be optionally substituted on carbon by one or more R²³; and wherein if said heterocyclyl contains an —NH— moiety that nitrogen may be optionally substituted by a group selected from R²⁴;
R²³ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
R²⁴ is selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆-alkyl)carbamoyl, benzyl, benzyloxycarbonyl, benzoyl and phenylsulphonyl;
or a pharmaceutically acceptable salt thereof.

2. The method according to claim 1 wherein A is a direct bond.

3. The method according to claim 1 wherein Ring C is phenyl, thienyl, pyridyl, thiazolyl.

4. The method according to claim 1 wherein R¹ is selected from hydrogen, C₁₋₆alkyl, C₁₋₆alkoxy, N,N—(C₁₋₆-alkyl)₂-amino, C₁₋₆alkylS(O)ₐ wherein a is 0 or carbocyclyl; wherein R¹ may be optionally substituted on carbon by one or more R⁸; wherein R⁸ is selected from halo or carbocyclyl.

5. The method according to claim 1 wherein R⁴ is hydrogen.

6. The method according to claim 1 wherein:
R² is C₁₋₆alkyl; wherein R² may be optionally substituted on carbon by one or more R¹⁰; R¹⁰ is selected from halo, hydroxy, carboxy, amino, C₁₋₆alkoxy, N,N—(C₁₋₆alkyl)₂amino, C₁₋₆alkanoylamino, N—(C₁₋₆alkyl)carbamoyl, N,N—(C₁₋₆alkyl)₂-carbamoyl or heterocyclyl; wherein R¹⁰ may be optionally substituted on carbon by one or more R¹⁹; and wherein if said heterocyclyl contains an —NH— moiety that nitrogen may be optionally substituted by a group selected from R²⁰;
R¹⁹ is selected from hydroxy or C₁₋₆alkoxy;
R²⁰ is C₁₋₆alkyl.

7. The method according to claim 1 wherein R³ is selected from halo, nitro, C₁₋₆alkyl or C₁₋₆alkoxy; wherein R³ may be optionally substituted on carbon by one or more R¹²; and R¹² is halo.

8. The method according to claim 1 wherein R⁵ is hydrogen or optionally substituted C₁₋₆alkyl; wherein said optional substituents are selected from one or more R¹⁴; and R¹⁴ is hydroxy.

9. The method according to claim 1 wherein:
R⁶ and R⁷ are independently selected from hydrogen, halo, nitro, cyano, amino, C₁₋₆alkyl, N—(C₁₋₆alkyl)amino, N,N—(C₁₋₆alkyl)amino, N—(C₁₋₆alkyl)carbamoyl, C₁₋₆alkoxycarbonyl or heterocyclyl; wherein R⁶ and R⁷ independently of each other may be optionally substituted on carbon by one or more R¹⁵; and wherein if said heterocyclyl contains an —NH— moiety that nitrogen may be optionally substituted by a group selected from R¹⁶;
or R⁶ and R⁷ together with the bond to which they are attached form a 6 membered carbocyclic ring or a 5 or 6 membered heterocyclic ring wherein said ring is fused to the pyrimidine ring in formula (I); wherein the double bonds of the resulting bicyclic ring may be further delocalised across the whole of the bicyclic ring; and wherein said carbocyclic ring or heterocyclic ring may be optionally substituted on carbon by one or more R¹⁷; and wherein if said heterocyclic ring contains an —NH— moiety that nitrogen may be optionally substituted by a group selected from R¹⁸;
R¹⁵ is selected from halo, hydroxy, amino, C₁₋₆alkoxy, N,N—(C₁₋₆alkyl)₂amino, carbocyclyl or heterocyclyl; wherein R¹⁵ may be optionally substituted on carbon by one or more R¹⁹; and wherein if said heterocyclyl contains an —NH— moiety that nitrogen may be optionally substituted by a group selected from R²⁰;
R¹⁷ is selected from halo, C₁₋₆alkyl or C₁₋₆alkoxy; wherein R¹⁷ may be optionally substituted on carbon by one or more R¹⁹;
R¹⁶ is C₁₋₆alkyl;
R¹⁸ is C₁₋₆alkanoyl;
R¹⁹ is selected from halo, hydroxy, C₁₋₆alkoxy or heterocyclyl; and wherein if said heterocyclyl contains an —NH— moiety that nitrogen may be optionally substituted by a group selected from R²⁴;
R²⁰ is C₁₋₆alkyl; and
R²⁴ is C₁₋₆alkyl.

10. The method according to claim 1 wherein n=0 or 1.

11. The method according to claim 1 wherein:
A is a direct bond;
Ring C is phenyl, thienyl, pyridyl, thiazolyl;
R¹ is selected from hydrogen, methyl, ethyl, isopropyl, 1-butyl, trifluoromethyl, cyclopropylmethyl, benzyl, methoxy, ethoxy, propoxy, isopropoxy, sec-butoxy, dimethylamino, methylthio or cyclopropyl;
R² is selected from methyl, ethyl, trifluoromethyl, hydroxymethyl, carboxymethyl, aminomethyl, methoxymethyl, morpholinomethyl, 1-hydroxyethyl, 2-hydroxyethyl, 1-carboxyethyl, 2-dimethylaminoethyl, 2-diethylaminoethyl, acetamidomethyl, 2-[N-methyl-N-(2-methoxyethyl)amino]ethyl, 2-[N-methyl-N-(2-hydroxyethyl)amino]ethyl, 2-(N-methylcarbamoyl)ethyl, 2-[N-(2-hydroxyethyl)carbamoyl]ethyl, 2-(N,N-dimethylcarbamoyl)ethyl, 2-morpholinoethyl, 2-pyrrolidin-1-ylethyl or 2-(1-methylpiperazin-4-yl)ethyl, 1-methyl-2-hydroxyethyl;
R³ is selected from fluoro, nitro, trifluoromethyl or methoxy;
R⁴ is hydrogen,
R⁵ is hydrogen, methyl or 2-hydroxyethyl;
R⁶ and R⁷ are independently selected from hydrogen, fluoro, chloro, bromo, nitro, cyano, amino, methyl, methylamino, ethylamino, propylamino, isopropylamino, dimethylamino, N-methyl-N-propylamino, N-ethylcarbamoyl, methoxycarbonyl, ethoxycarbonyl, butoxycarbonyl, morpholino, pyrrolidinyl or piperazinyl; wherein R⁶ and R⁷ independently of each other may be optionally substituted on carbon by one or more R¹⁵; and wherein said piperazinyl may be optionally substituted on nitrogen by a group selected from R¹⁶;
or R⁶ and R⁷ together with the pyrimidine to which they are attached form a bicyclic ring selected from quinazolinyl, thieno[3,2-d]pyrimidinyl, thieno[2,3-d]pyrimidinyl, 1H-pyrazolo[3,4-d]pyrimidinyl, thieno[3,4-d]pyrimidinyl, pyrido[2,3-d]pyrimidinyl, 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl; and wherein said bicyclic ring may be optionally substituted on carbon by one or more R¹⁷; and wherein said 5,6,7,8-tetrahydro-pyrido[4,3-d]pyrimidinyl, 5,6,7,8-tetrahydro-pyrido[2,3-d]pyrimidinyl or 5,6,7,8-tetrahydro-pyrido[3,4-d]pyrimidinyl may be optionally substituted on nitrogen by a group selected from R¹⁸;
R¹⁵ is selected from fluoro, hydroxy, amino, ethoxy, dimethylamino, phenyl, pyrrolidinyl, piperazinyl or morpholino, wherein R¹⁵ may be optionally substituted on carbon by one or more R¹⁹; and wherein said piperazinyl may be optionally substituted on nitrogen by a group selected from R²⁰;
R¹⁶ is methyl;
R¹⁷ is selected from fluoro, chloro, methyl, methoxy, ethoxy or propoxy; wherein R¹⁷ may be optionally substituted on carbon by one or more R¹⁹;
R¹⁸ is acetyl;
R¹⁹ is selected from fluoro, hydroxy, methoxy, piperazinyl, pyrrolidinyl or morpholino;
and wherein said piperazinyl may be optionally substituted on nitrogen by a group selected from R²⁴;
R²⁰ is methyl;
R²⁴ is methyl;
n=0 or 1;
or a pharmaceutically acceptable salt thereof.

12. The method of claim 1, wherein the compound of formula (I) is selected from:
(2R)-2-({4-[(5-cyclopropyl-1H-pyrazol-3-yl)amino]-5-fluoropyrimidin-2-yl}amino)-2-(4-fluorophenyl)ethanol;
5-bromo-N⁴-(5-cyclopropyl-1H-pyrazol-3-yl)-N²-[(1S)-1-(4-fluorophenyl)ethyl]pyrimidine-2,4-diamine;
(2R)-2-({5-chloro-4-[(5-cyclopropyl-1H-pyrazol-3-yl)amino]pyrimidin-2-yl}amino)-2-(4-fluorophenyl)ethanol;
(2R)-2-({5-chloro-4-[(3-isopropoxy-1H-pyrazol-5-yl)amino]pyrimidin-2-yl}amino)-2-(4-fluorophenyl)ethanol;
(3S)-3-({5-chloro-4-[(5-cyclopropyl-1H-pyrazol-3-yl)amino]pyrimidin-2-yl}amino)-3-(4-fluorophenyl)-N-methylpropanamide;
(3S)-3-({4-[(5-Cyclopropyl-1H-pyrazol-3-yl)amino]-5-chloropyrimidin-2-yl}amino)-3-(4-fluorophenyl)propan-1-ol;
2-({5-chloro-2-{[(1S)-1-(4-fluorophenyl)ethyl]amino}-6-[(5-isopropoxy-1H-pyrazol-3-yl)amino]pyrimidin-4-yl}amino)propane-1,3-diol;
2-[(5-chloro-6-[(5-cyclopropyl-1H-pyrazol-3-yl)amino]-2-{[(1S)-1-(4-fluorophenyl)ethyl]amino}pyrimidin-4-yl)amino]propane-1,3-diol;
5-chloro-N⁴-(5-cyclopropyl-1H-pyrazol-3-yl)-N²-[(1S)-(4-fluoro-phenyl)-ethyl]-6-(4-methyl-piperazin-1-yl)-pyrimidine-2,4-diamine;
5-Chloro-N⁴-(5-cyclopropyl-1H-pyrazol-3-yl)-N²-[(1S)-1-(4-fluorophenyl)ethyl]pyrimidine-2,4-diamine;
(2R)-2-({4-[(5-cyclopropyl-1H-pyrazol-3-yl)amino]-7-fluoroquinazolin-2-yl}amino)-2-(4-fluorophenyl)ethanol; and
2-[(5-chloro-6-[(5-cyclopropyl-1H-pyrazol-3-yl)amino]-2-{[(1R)-1-(4-fluorophenyl)-2-hydroxyethyl]amino}pyrimidin-4-yl)amino]propane-1,3-diol;
or a pharmaceutically acceptable salt thereof.

13. The method according to claim 1, wherein the method comprises therapeutic treatment of the subject.

14. The method according to claim 1, wherein the method comprises prophylactic treatment of the subject.

15. The method according to claim 1 wherein said pain is selected from pain caused by chemical, mechanical, radiation, thermal, infectious or inflammatory tissue trauma or cancer.

16. The method according to claim 1 wherein said pain is posttraumatic pain, headache and migraine, various arthritic and inflammatory conditions such as osteo and rheumatoid arthritis, myofascial and low back pain associated with chronic inflammation, bone diseases, cell proliferation such as cancers (solid tumors and leukemia).

17. The method according to claim 1 wherein said pain is of central or peripheral origin, such as trigeminal neuralgia, postherpetic neuralgia (PHN), painful diabetic mono/poly neuropathy, and pain associated with nerve damage, spinal cord injury central post stroke, multiple sclerosis and Parkinson's disease.

18. The method according to claim 1 wherein said pain is of visceral origin such as caused by ulcer, dysmenorrhea, endometriosis, IBS and dyspepsia.

19. The method according to claim 1, wherein the daily dose of the compound of formula (I) is in the range of from about 0.1 mg to about 1000 mg of active substance.

20. The method according to claim 19, wherein the daily dose of compound of formula (I) is in the range of from about 1 mg to about 750 mg of active substance.

21. The method according to claim 20, wherein the daily dose of compound of formula (I) is in the range of from about 1 mg to about 500 mg of active substance.

22. A pharmaceutical formulation for use in the prophylactic and/or therapeutic treatment of pain, said formulation comprising a compound of formula (I) as the active substance in optional admixture with a pharmaceutically acceptable adjuvant, diluent or carrier.

23. (canceled)

24. The method according to claim 1 wherein said pain is selected from pain caused by chemical, mechanical, radiation, thermal, infectious or inflammatory tissue trauma or cancer, is posttraumatic pain, headache and migraine, various arthritic and inflammatory conditions such as osteo and rheumatoid arthritis, myofascial and low-back pain associated with chronic inflammation, bone diseases, cell proliferation such as cancers (solid tumors and leukemia), is pain of central or peripheral origin, such as trigeminal neuralgia, postherpetic neuralgia (PHN), painful diabetic mono/poly neuropathy, pain associated with nerve damage, spinal cord injury, central post stroke, multiple sclerosis and Parkinson's disease, is pain of visceral origin such as caused by ulcer, dysmenorrhea, endometriosis, IBS and dyspepsia.

25. (canceled)
